(19) United States
(12) Patent Application Publication
(10) Pub. No.: US 2005/0043150 A1
(43) Pub. Date: Feb. 24, 2005

(54) Exercise foot harness

(75) Inventors: Jeffrey A. Nitta, Broomfield, CO (US); Dominic Lovely, Denver, CO (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: Nautilus, Inc., Vancouver, WA (US)

(21) Appl. No.: 10/911,141

(22) Filed: Aug. 3, 2004

Related U.S. Application Data
(60) Provisional application No. 60/493,015, filed on Aug. 5, 2003.

(51) Int. Cl. A63B 23/10; A61F 5/00

(52) U.S. Cl. 482/79; 602/27

(57) ABSTRACT

An exercise foot harness adapted to be secured to either foot or shoe of a user. The exercise foot harness includes and interconnection of a toe strap, a midstrap, and a heel strap. The toe strap includes a coupling member so that the toe strap may be secured around the toe area of an athletic shoe. The toe strap also includes one or more coupling points where a user may secure a resistance device, such as a weight cable of a home gym or other exercise machine. The heel strap includes a coupling member so that the heel strap may be secured around the heel area of an athletic shoe. The heel strap also includes one or more coupling points where a user may secure a resistance device. Finally, the midstrap is used to secure the exercise foot harness around the midportion of an athletic shoe. In one particular arrangement, coupling points are provided at the top of the toe, the bottom of the toe, the left and right side, and the heel so that a user may connect a resistance device or devices to any number of combinations of coupling points and perform numerous different exercises.

### CROSS-REFERENCE TO RELATED APPLICATIONS

This application claims priority to U.S. Provisional Patent Application No. 60/493,015 titled “Exercise Foot Harness” filed Aug. 5, 2003, which is hereby incorporated by reference herein.

### FIELD OF THE INVENTION

The present invention generally relates to the field of exercise and exercise devices, and more particularly involves an exercise foot strap with one or more coupling points adapted to connect with a weight training machine or other exercise device.

### BACKGROUND OF THE INVENTION

Weight training machines often include pulley and cable arrangements coupled with a weight stack or other resistance mechanism. The free end of the cable, distal the weight stack connection, may be connected to various pivotal arm configurations to provide various exercises ranging from bench press, bicep curls, to sitting rows. The free end of the cable may also be coupled with a handle so that the user may directly actuate the resistance mechanism by grasping the handle with his hand and performing various arm and body movements.

The present invention involves an exercise foot harness providing a plurality of coupling points for operably attaching the foot harness to a resistance mechanism whereby a user may secure the foot harness to his foot and perform a wide variety of foot, leg, and lower body exercises by actuating the resistance mechanism.

### SUMMARY OF THE INVENTION

One aspect of the invention involves an exercise foot harness comprising a toe strap, a connecting member coupled with the toe strap, and a midstrap coupled with the connecting member. The toe strap defines a first segment and a second segment, the toe strap further comprises at least one coupling member adapted to interconnect the first segment and the second segment, the toe strap further comprises at least one first coupling point. The midstrap defines a third segment and a fourth segment, the midstrap further comprises at least one second coupling member adapted to interconnect the third segment and the fourth segment. The heel strap defines a fifth segment and a sixth segment, the heel strap further comprises at least one third coupling member adapted to interconnect the fifth segment and the sixth segment, the heel strap further comprises at least one second coupling point.

The toe strap is configured to be secured around a toe portion of a typical athletic shoe. The midstrap is configured to be secured around a midportion of a typical athletic shoe. And, the heel strap is configured to be secured around a heel portion of a typical athletic shoe.

The coupling members may include a buckle, a ladder lock buckle, a hook and loop fastener, a clip, and a snap lock.

In one particular aspect of the invention, the toe strap further comprises a first adjustment segment supporting at least one of the at least one first coupling point. The first adjustment segment may be arranged adjacent the second segment. In another particular aspect of the invention, the at least one coupling member comprises a first ladder lock buckle adapted to interconnect the second segment and the first adjustment segment and further comprising a second ladder lock buckle adapted to interconnect the first adjustment segment and the first segment. The first ladder lock buckle and the second ladder lock buckle may be arranged to provide adjustment for the at least one first coupling point.

In another particular aspect of the invention, the heel strap further comprises a seventh segment supporting at least one of the at least one second coupling point. The seventh segment may be arranged adjacent the sixth segment. In another particular aspect of the invention, the at least one third coupling member comprises a third ladder lock buckle adapted to interconnect the sixth segment and the seventh segment and further comprising a fourth ladder lock buckle adapted to interconnect the seventh segment and the fifth segment. The third ladder lock buckle and the fourth ladder lock buckle may be arranged to provide adjustment for the at least one second coupling point.

In another particular aspect of the invention, the at least one first coupling point comprises a top toe coupling point and a bottom toe coupling point. Additionally, the at least second coupling point comprises a left side coupling point, a right side coupling point, and a heel coupling point. In another particular aspect of the invention, the top toe coupling point is arranged generally along the top of the toe region of a typical athletic shoe when the exercise foot harness is deployed. Additionally, the bottom toe coupling point is arranged generally along the bottom of the toe region of a typical athletic shoe when the exercise foot harness is deployed. Further, the left side coupling point is arranged generally along the left side of a typical athletic shoe when the exercise foot harness is deployed. Still further, the right side coupling point is arranged generally along the right side of a typical athletic shoe when the exercise foot harness is deployed. Finally, the heel coupling point is arranged generally along the heel of a typical athletic shoe when the exercise foot harness is deployed. The coupling points may include a ring, a D-ring, an O-ring, a strap, a hook, a loop, a carabiner, an eyelet, or other mechanism to couple the exercise foot harness with a resistance device or a cable connected with resistance device.

Another aspect of the invention involves an exercise foot harness adapted for being secured to an athletic shoe, the athletic shoe including a toe region, a heel region, and a mid region between the toe region and the heel region. The exercise foot harness comprises means for attaching at least one exercise strap to the toe region of an athletic shoe; means for attaching at least one exercise strap to the heel region of an athletic shoe; and means for securing the exercise foot harness to the mid region of an athletic shoe. The exercise foot harness may further comprise means for interconnecting the means for attaching at least one exercise strap to the toe region of an athletic shoe with the means for securing the exercise foot harness to the mid region of an athletic shoe. Additionally, the exercise foot harness may further comprise means for attaching at least one exercise strap to the heel region of an athletic shoe is coupled with the means for securing the exercise foot harness to the mid region of an athletic shoe.

Another aspect of the invention involves an exercise foot harness comprising a toe member adapted to engage a toe portion of a typical athletic shoe; a heel member adapted to engage a heel portion of a typical athletic shoe; at least one interconnecting member coupled between the toe member and the heel member; and a plurality of coupling points. The exercise foot harness may further comprise a midmember adapted to engage a midportion of a typical athletic shoe.

In one particular aspect, the midmember comprises a first strap adapted to engage the arch region of a typical athletic shoe. Additionally, the midmember may comprise a second strap adapted to engage the lace region of a typical athletic shoe. In another particular aspect, the at least one interconnecting member comprises a first side strap coupled between the toe member and the heel member and a second side strap coupled between the heel member and the toe member.

In various aspects, the toe member/strap, heel member/strap, interconnecting member/strap and other members and straps may include length adjustment features, such as buckling arrangements, hook and loop fasteners, snaps, clips, and the like.

In one particular aspect, the at least one toe member comprises a molded cavity adapted to engage the toe portion of the typical athletic shoe. Further, the at least one heel member comprises a second molded cavity adapted to engage the heel portion of a typical athletic shoe. Each cavity may include an open end where the front end or rear end, respectively, of an athletic shoe protrudes therefrom.

Another aspect of the invention involves an exercise foot harness comprising a first member adapted to engage a front portion of a typical athletic shoe; a second member adapted to engage a rear portion of a typical athletic shoe; and at least one coupling point. In one particular aspect, the first member comprises a toe member with a first coupling point and a second coupling point. In another particular aspect, the second member comprises a heel member with a third coupling point and a fourth coupling point.

The first member may comprise a midmember. The first member and the second member may be provided by a single strap. The exercise foot harness may further include at least one interconnecting member between the first member and the second member.

In another particular aspect, the third member is adapted to engage a rear portion of a typical athletic shoe. The front portion of a typical athletic shoe comprises a toe area, in one example. The rear portion of a typical athletic shoe comprises the lacing area, in one example. The rear portion of a typical athletic shoe may also comprise the heel area, in one example. Moreover, the rear portion of a typical athletic shoe may comprise a region between and including the lacing area and the heel area.

### BRIEF DESCRIPTION OF THE DRAWINGS

FIG. 1 illustrates an isometric view of a user with one particular implementation of an exercise foot harness, in accordance with aspects of the present invention, deployed over the shoe of a user's right foot and being used to exercise.

FIG. 2 is a right upper side perspective view of one embodiment of the exercise foot harness, in accordance with aspects of the present invention.

FIG. 3 is a right rear side perspective view of the exercise foot harness shown in FIG. 2.

FIG. 4 is a right lower side perspective view of the exercise foot harness shown in FIG. 2.

FIG. 5 is a left lower side perspective view of the exercise foot harness shown in FIG. 2.

FIGS. 6-8 illustrate a bottom view, a top view (also shows hidden line of foot illustrating where a user might place his or her foot to strap on the harness), and an isometric view of the foot harness shown in FIG. 2 in an undeployed arrangement, such as before the exercise foot strap is connected to the shoe or foot of a user.

FIG. 9 is an isometric view of an alternative exercise foot harness, in accordance with one embodiment of the present invention.

FIGS. 10-12 illustrate a top view (how the harness is laid-out on a floor before securing to the shoe), a bottom view, and an isometric view, respectively, of an alternative embodiment of the foot harness, in accordance with the present invention. The embodiment of FIGS. 10-12 employing ladder lock type buckles arranged to facilitate the location of any of a plurality of coupling points.

FIG. 13 is a right upper side perspective view of the embodiment of the exercise foot harness shown in FIGS. 10-12 deployed over a typical athletic shoe.

FIG. 14 is a right rear side perspective view of the exercise foot harness shown in FIGS. 10-12 deployed over a typical athletic shoe.

FIG. 15 is a right lower side perspective view of the exercise foot harness shown in FIGS. 10-12 deployed over a typical athletic shoe.

FIG. 16 is a left lower side perspective view of the exercise foot harness shown in FIGS. 10-12 deployed over a typical athletic shoe.

FIGS. 17 and 18 illustrate a top view and a perspective view, respectively, of an alternative exercise foot harness, in accordance with aspects of the present invention, deployed on a typical athletic shoe.

FIGS. 19 and 20 illustrate a right side view and a perspective view, respectively, of an alternative exercise foot harness, in accordance with aspects of the present invention, deployed on a typical athletic shoe.

FIGS. 21 and 22 illustrate a right side view and a perspective view, respectively, of an alternative exercise foot harness, in accordance with aspects of the present invention, deployed on a typical athletic shoe.

FIGS. 23 and 24 illustrate a right side view and a perspective view, respectively, of an alternative exercise foot harness, in accordance with aspects of the present invention.

FIGS. 25 and 26 illustrate a right side view and a perspective view, respectively, of another alternative embodiment of an exercise foot harness in accordance with the present invention.

FIGS. 27 and 28 illustrate a top view and a perspective view, respectively, of an alternative exercise foot harness, in accordance with aspects of the present invention, deployed on conventional athletic shoes.

FIGS. 29 and 30 illustrate a top view and a perspective view, respectively, of yet another alternative exercise foot harness in accordance with the present invention, deployed over a typical athletic shoe.

FIGS. 31 and 32 illustrate a side right view and a perspective view, respectively, of an exercise foot harness, in accordance with one embodiment of the present invention, deployed over a typical athletic shoe.

FIGS. 33 and 34 illustrate a right side view and a perspective view, respectively, of an alternative embodiment of an exercise foot harness, in accordance with aspects of the present invention, deployed over an athletic shoe.

FIGS. 35 and 36 illustrate a right side view and a perspective view, respectively, of an alternative exercise foot harness, in accordance with one example of the present invention, deployed over an athletic shoe.

FIGS. 37 and 38 illustrate a right side view and perspective view, respectively, of an alternative exercise foot harness, in accordance with one example of the present invention, deployed over an athletic shoe.

FIGS. 39 and 40 illustrate a right side view and a perspective view, respectively, of yet another alternative exercise foot harness, in accordance with aspects of the present invention, deployed over an athletic shoe.

FIGS. 41 and 42 illustrate a top view and a perspective view, respectively, of the exercise foot harness shown in FIG. 43 attached to a typical athletic shoe.

FIG. 43 is a front view of an undeployed exercise foot harness, in accordance with aspects of the present invention.

FIGS. 44 and 45 illustrate a top view and a perspective view of the exercise foot strap shown in FIG. 43 attached to a typical athletic shoe in a different arrangement than that shown in FIGS. 41 and 42.

FIGS. 46 and 47 illustrate a top view and a perspective view, respectively, of an example of an exercise foot harness shown in FIGS. 48 and 49, deployed on a typical athletic shoe.

FIGS. 48 and 49 illustrate a perspective view and a top view, respectively, of an alternative exercise foot harness, in accordance with aspects of the present invention.

FIGS. 50 and 51 illustrate a side view and a perspective view, respectively, of an alternative exercise foot strap shown in FIGS. 52 and 53, deployed on a typical athletic shoe.

FIGS. 52 and 53 illustrate a right side perspective view and a top view, respectively, of an alternative exercise foot harness, in accordance with aspects of the preset invention.

FIGS. 54-55 illustrate a top view and a perspective view, respectively, of an alternative exercise foot harness shown in FIG. 56, deployed on a typical athletic shoe.

FIG. 56 illustrates a perspective view of an alternative exercise foot harness, in accordance with aspects of the present invention.

FIGS. 57 and 58 illustrate a right side view and a perspective view of an alternative exercise foot harness in accordance with aspects of the present invention, deployed over a conventional athletic shoe.

### DETAILED DESCRIPTION OF EMBODIMENTS OF THE INVENTION

Aspects of the present invention involve an exercise foot harness that has one or more coupling points by which a user may connect his or her foot or feet to a resistance device in order to perform foot, ankle, leg, and other exercises. In one particular implementation, when the foot harness is secured to the foot, one or more coupling rings are located at the front of the foot, each side of the foot, at the heel of the foot, and at the bottom of the foot. With such an arrangement, the user may connect the cable of a resistance device, such as a weigh stack, to one of the coupling rings positioned at the various regions of the foot, and exercise his or her foot, ankle, etc., by actuating the resistance device. The coupling points are positioned on the exercise foot harness located so that numerous different exercises may be performed with the use of the foot harness.

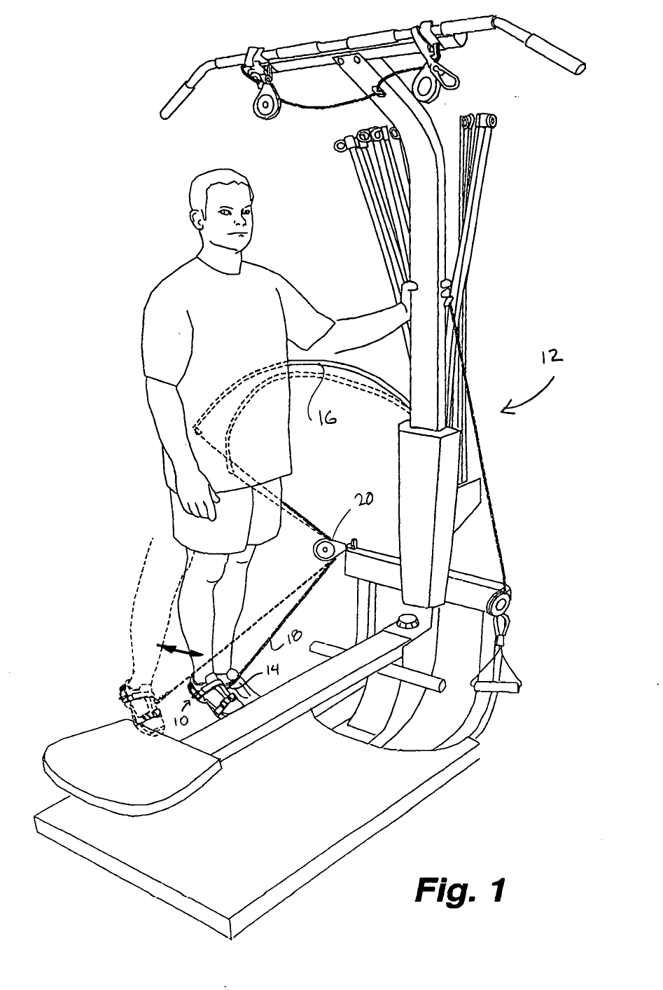

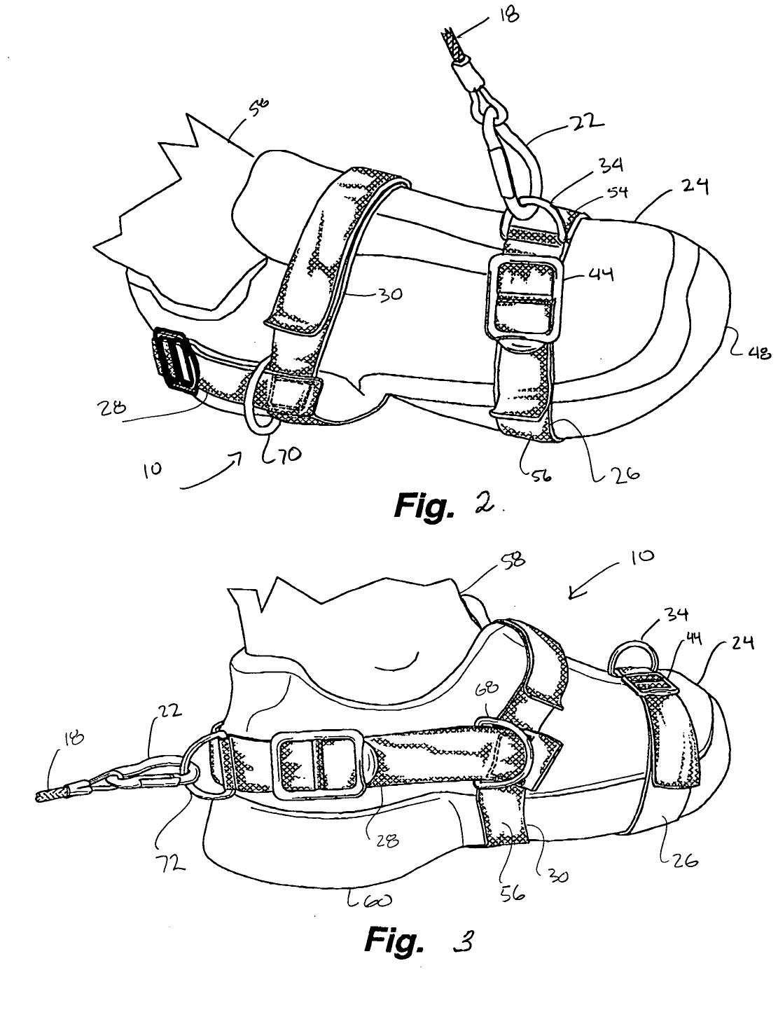

FIG. 1 illustrates an isometric view of a user with one particular implementation of an exercise foot harness 10, in accordance with one embodiment of the present invention, attached to the user's right foot. Some embodiments of the foot harness are universal, so that they may be fitted to the right or left foot of the user. The user is shown exercising with a Bowflex Ultimate™ exercise machine 12. One of the coupling points 14 on the foot harness is connected with a Power Rod™ 16 of the exercise machine. Although shown here being used with a Bowflex™ exercise machine, the foot harness may be employed with just about any type of exercise device, such as any of the numerous types of commercial and home gyms that employ weight stacks. Referring again to FIG. 1, a cable 18 is connected with the power rod via a pulley 20. The left side coupling point of the foot harness is connected with the cable, such as with a carabiner type connector 22 (FIG. 2).

In FIG. 1, the user is shown performing a hip abduction exercise, which involves lateral movement of the thigh outward with the hip being kept generally straight. Particularly, the user is shown standing generally straight, with both feet on the ground and the legs generally parallel. The user is facing the bench portion of the exercise machine 12. In hidden line, the user is shown with his right leg extended outwardly from the body. When extending the leg outwardly, the force imparted by the user is coupled to the power rod 16 through the exercise foot harness 10, the cable 18 and the pulley 20, causing the power rod to bend in an arc. It can be readily seen that by connecting the resistance means with the various coupling points of the exercise foot harness any number of foot and leg exercised may be performed. Moreover, the user may use a foot harness on both the right and left foot, and perform any number of different exercises involving muscles groups of the feet, ankles, legs, or other parts of the body, alone or in combination.

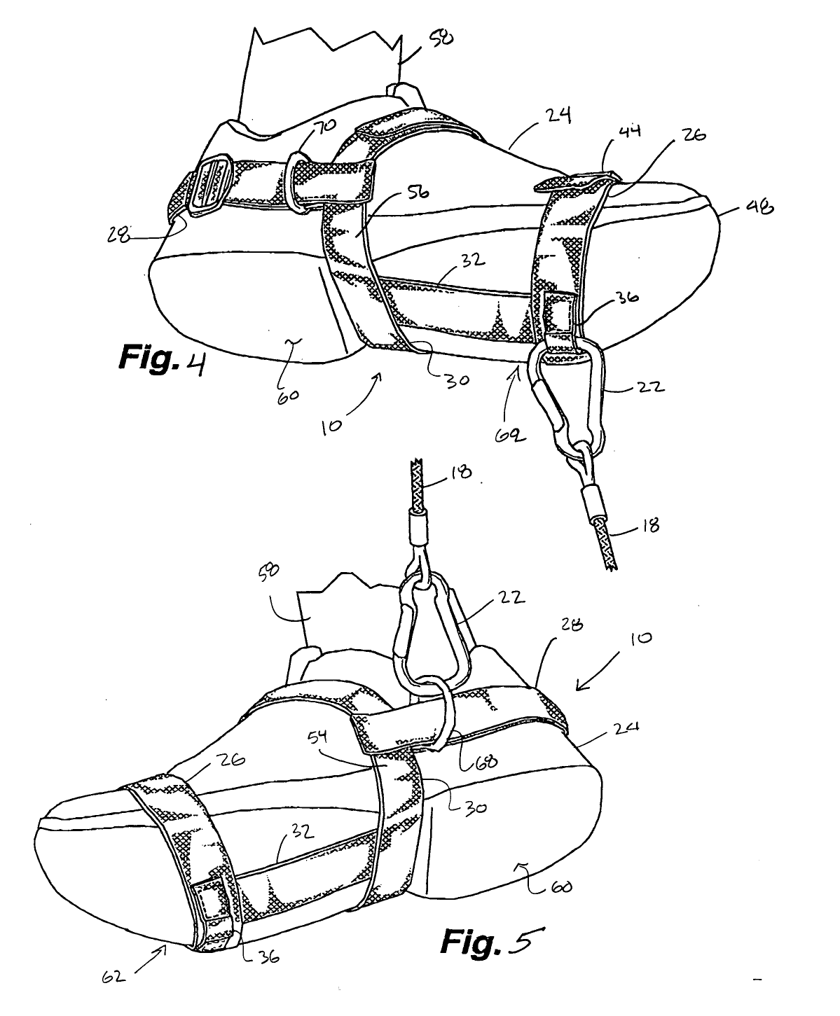

FIGS. 2-5 illustrate various isometric views of one particular embodiment of the exercise foot harness 10 deployed over a shoe 24 worn by a user. Particularly, FIG. 2 is a right upper side perspective view of one embodiment of the exercise foot harness. FIG. 3 is a right rear side perspective view of the exercise foot harness 10 shown in FIG. 2. FIG. 4 is a right lower side perspective view of the exercise foot harness shown in FIG. 2. Finally, FIG. 5 is a left lower side perspective view of the exercise foot harness shown in FIG. 2. In addition, FIGS. 6-8 illustrate a top view, a bottom view, and an isometric view of the foot harness shown in FIG. 2 in an undeployed arrangement, such as before the exercise foot harness 10 is connected to the shoe or foot of a user. Typically, the exercise foot strap is employed over a shoe worn by the user; however, it may be employed directly over the foot or a sock. As used herein, the phrases “exercise foot harness,” “foot harness,” “harness,” and “exercise harness” are used interchangeably.

Referring now to FIGS. 2-8, in one implementation, the foot harness 10 comprises a toe strap 26, a heel strap 28, and a midstrap 30. The midstrap is connected with the heel strap. The midstrap is also connected with the toe strap via a connecting plate or strap 32. In some implementations, the various straps may be nylon, neoprene or plastic. Referring particularly to FIGS. 6-8, views of a foot harness before deployment on a user's shoe are shown. As used herein, the terms “front,” “rear,” “left,” and “right” refer to the front, left, etc. perspective of a user. At the front of the foot harness, the toe strap includes a top coupling member 34 connected to a left portion 35 of the toe strap and a bottom coupling member 36. In the particular implementation of FIGS. 6-8, the coupling members are D-rings. Other coupling arrangements, such as an O-ring, fabric sewn to define a loop (see, for example, the bottom coupling point 36 of the harness 10 of FIGS. 4-5, a carabineer, a clasp, a catch, etc. may be used in place of the D-rings. The D-rings include an arcuate section 38 and a straight section 40. The D-rings are connected with the toe strap 26 using a segment of strap material 42 deployed over the straight portion of the D-ring, and sewn to the strap on either side of the straight portion. In this manner, the strap defines a cylinder-like area to secure the D-ring so that the arcuate section may pivot about the straight section. Note, other D-rings are pivotally connected to the harness in a similar fashion.

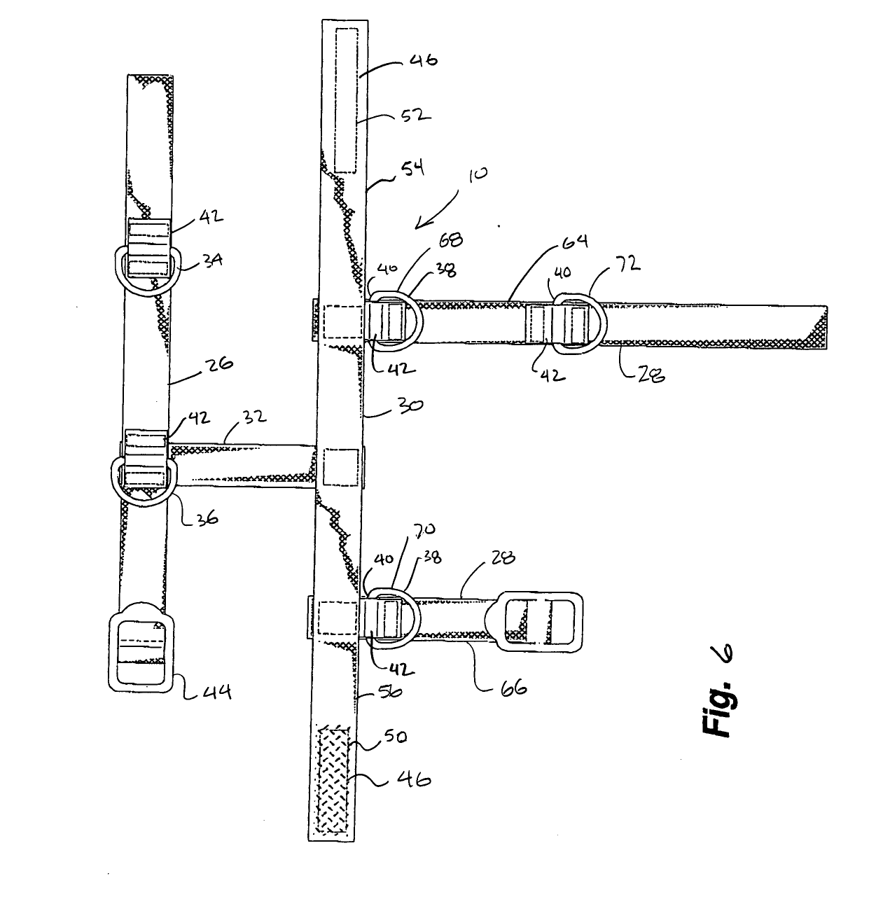

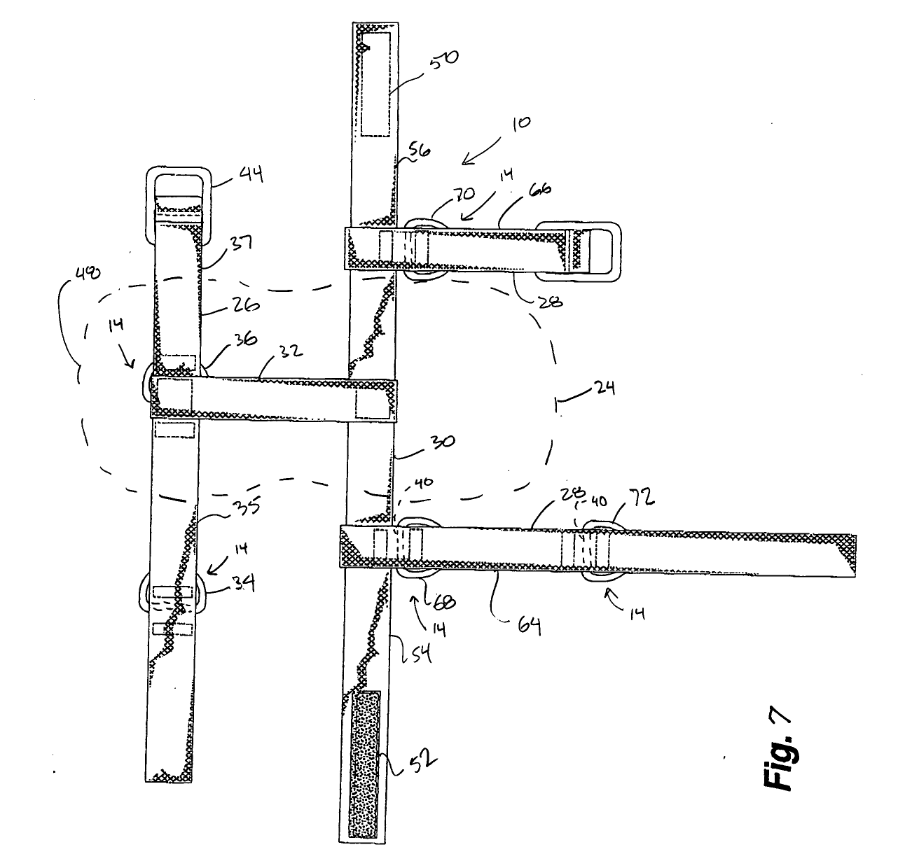

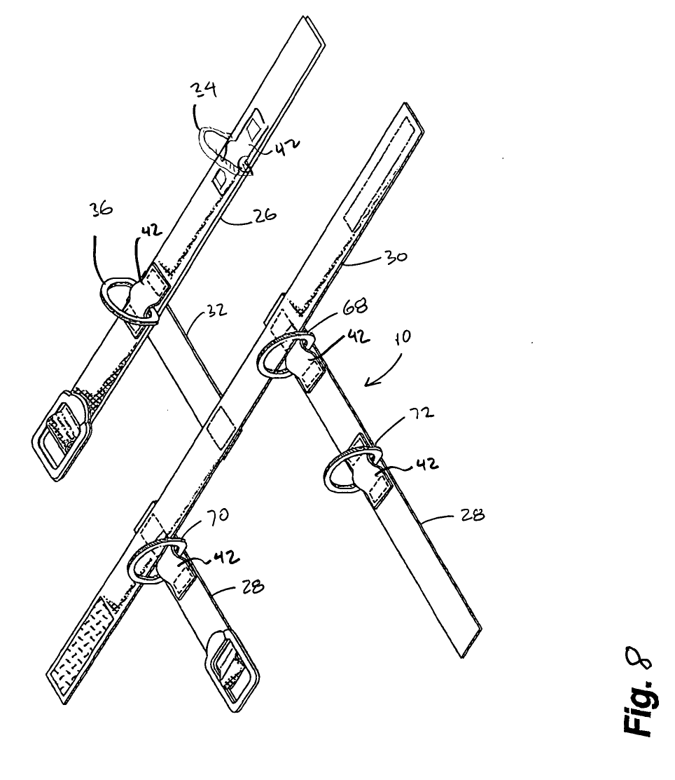

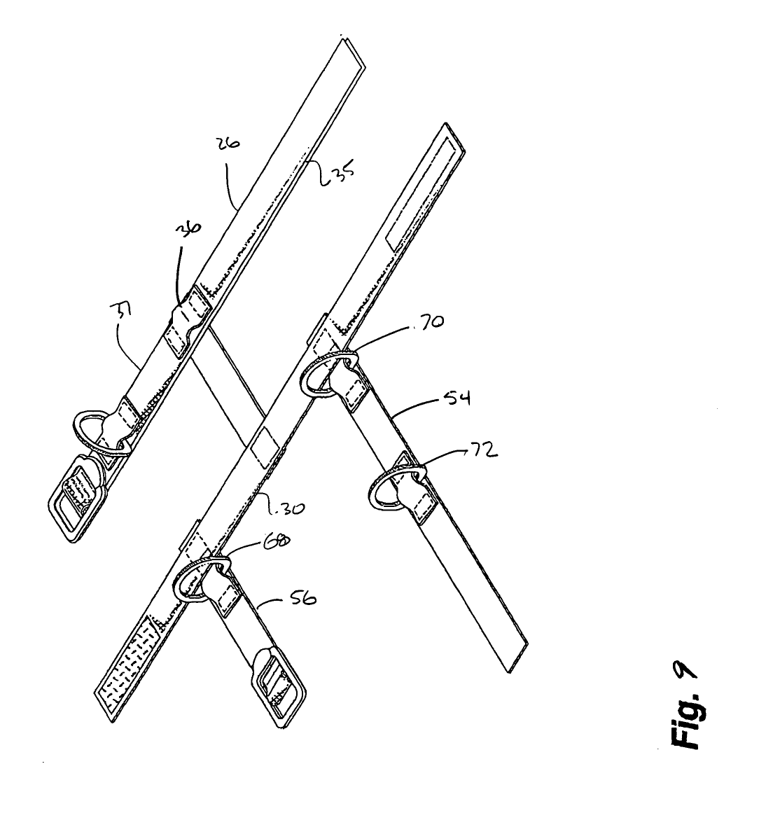

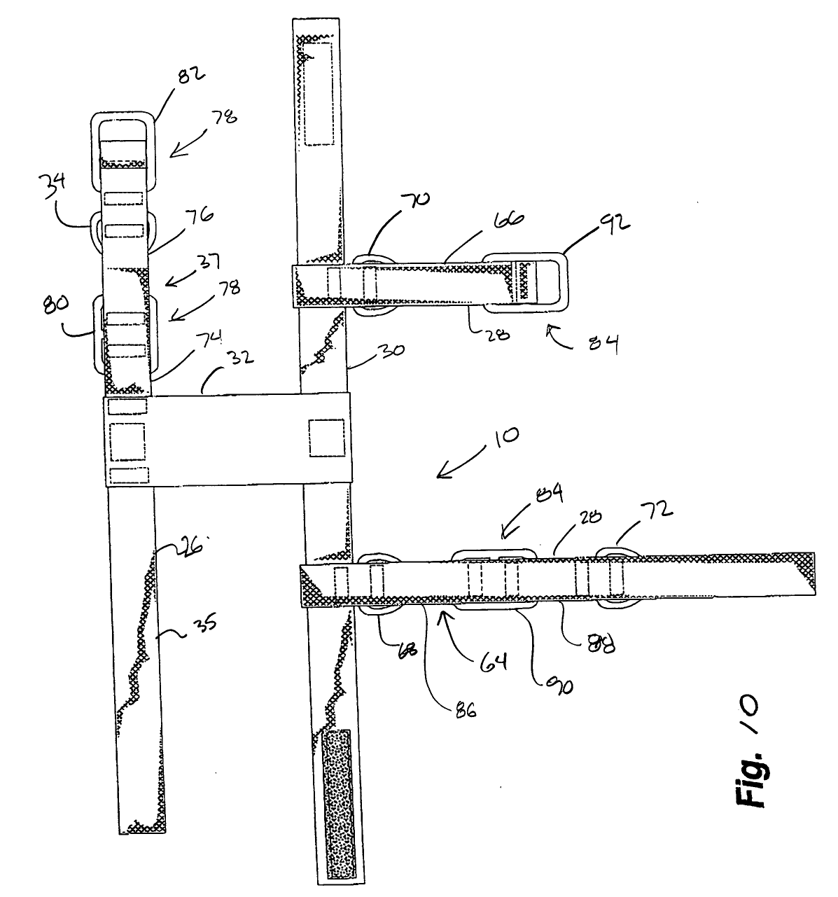

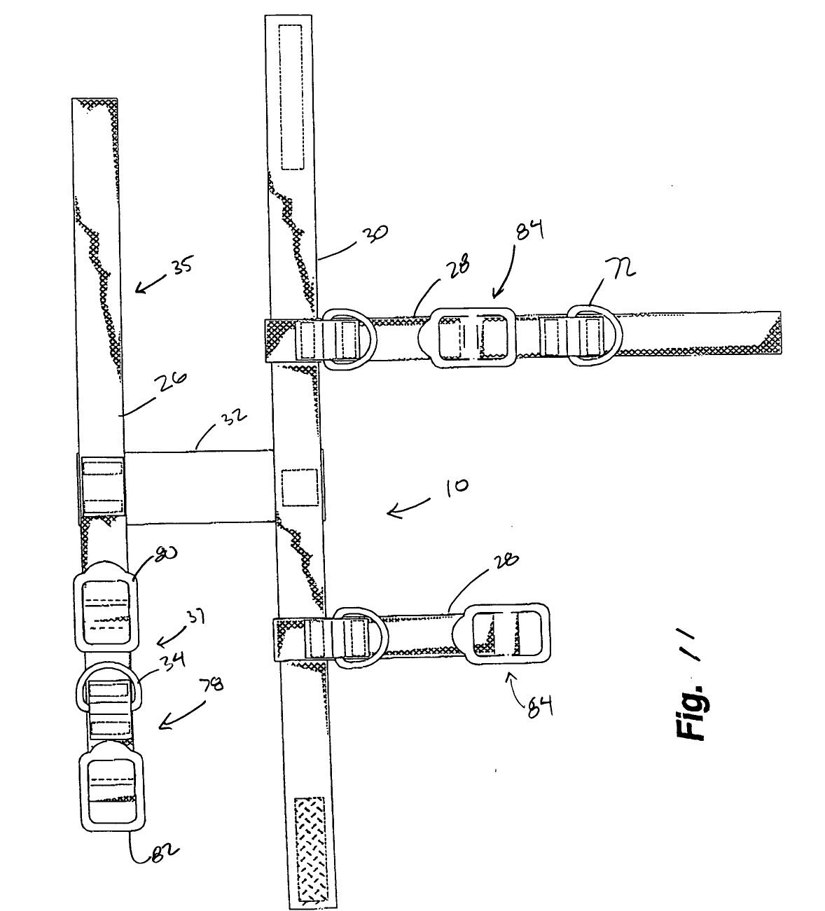

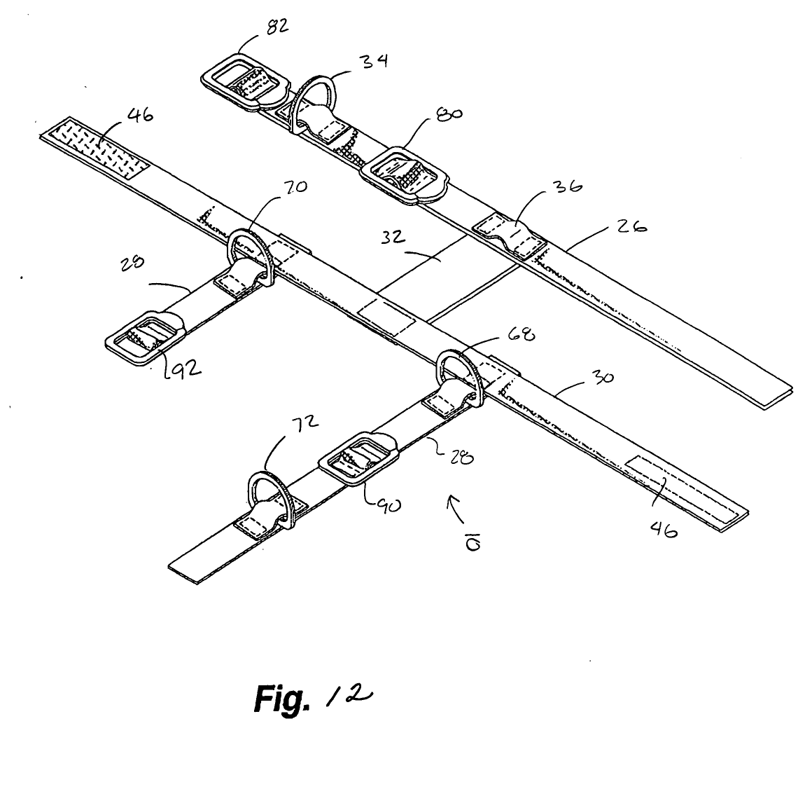

The toe strap 26 also includes a buckle 44 at or near one end of the strap. In the example of FIG. 7, the buckle is connected to a right portion 37 of the toe strap. To secure the toe strap around the front of the foot in the area of the toe, the opposing free end of the strap may be fed through the buckle and secured to the buckle. As shown best in FIG. 2, secured as such, the toe strap circumscribes the toe area of the shoe and is tightened thereto. As will be discussed with reference to other embodiments of the foot harness (see, for example, FIG. 11), two buckles may also be employed to assist in properly locating the D-rings at appropriate locations with respect to the user's foot. In some of the embodiments shown herein, a ladder lock type buckle is employed. In addition, other types of securing means besides a buckle may be employed, such as a hook and loop type fastener (e.g., Velcro™), a snap or plurality of snaps, a clasp, a pinch connector, and others. For example, the midstrap 30 is fastened around the shoe using a hook and loop fastener. The toe strap buckle and other fasteners and buckles described herein, provide numerous adjustment points, so that the foot harness may be used and secured to different shoe sizes and types.

Referring to FIGS. 2-5, when the foot harness 10 is operably secured to a user's shoe 24, the top coupling member 34, e.g., the top D-ring, is arranged along the top of the user's shoe, just rearward of the front 48 of the shoe, and generally around the midpoint of the width of the shoe. The top coupling member provides a top front coupling point. The bottom coupling member 36, e.g., the bottom D-ring, is arranged along the bottom of the user's shoe, also just rearward of the front of the shoe and generally around the midpoint of the width of the shoe. The bottom coupling member provides a bottom coupling point. The top and bottom coupling points (and other coupling points discussed below) provide a means by which a user may secure a resistance mechanism, e.g., a cable operably connected to a weight stack or Power Rod™, to perform any number of exercises. Particularly, examples of exercises that may be performed via the top coupling member 34 include a standing hip extension and a lying circumduction. Examples of exercises that may be performed via the bottom coupling member 36 include a lying hip flexion with knee extension and a ankle dorsi flexion.

The connecting strap 32 provides a connection between the toe strap 26 and the midstrap 30. Other implementations shown herein provide a coupling plate in place of the coupling strap. In addition, as will be discussed in more detail below, the connection strap, provides a locating function by which the user may properly position his or her foot prior to securing the exercise harness to his or her foot. In the embodiments shown herein, the connecting plate is a rectangular section of polymer, such as “plastic,” polyvinyl, strap webbing (like the straps) and the like. In the implementations shown in FIGS. 2-8, the front end of the connecting strap is sewn to the toe strap such that the connecting strap is at about a right angle with respect to the toe strap. The rear end of the connecting strap is sewn to the midstrap such that the connecting plate is also at about a right angle with respect to the midstrap. Connected between the toe and midstrap in this manner, the toe and midstrap are arranged generally parallel when they are laid out flat on the floor, such as is shown in FIG. 7. Some users will desire to arrange the exercise harness on the floor as shown in FIG. 7 before securing the harness to his or her foot/shoe.

Other materials and configurations, such as metals, fabrics, straps, flexible resilient cords, etc., may also be employed to provide a connection between the toe strap and midstrap. In addition, the various straps (26, 28, 30) and the connecting member 32 of the foot harness 10 may be fabricated form a single piece of strap material, such as by cutting or stamping the entire harness configuration from a single section of strap material. In such a manufacturing process, it might be desirable to seal the edges of the strap portions by sewing in a thread along the edges of the straps or melting or heat sealing the edges of the straps, in a case where a meltable material is employed for the straps. Moreover, the straps may be fabricated from other materials as well, such as cords, flexible resilient materials, polymers, etc., and combinations of materials (e.g., straps, cords, and polymers). Finally, as will be discussed in more detail below, the harness may include shaped pieces, stiffeners, and the like, to facilitate fitting the harness to a shoe or foot.

When the foot harness is connected with a user's foot, the midstrap 30 is located rearwardly of the toe strap. Along one end region of the midstrap, a hook portion 50 of the hook and loop fastener 46 is sewn to the strap. Along the other end region of the midstrap, a loop portion 52 of the hook and loop fastener is sewn to the strap. The midstrap generally includes a left portion 54 and a right portion 56. When the midstrap is arranged on a user's foot or shoe, the left portion of the midstrap is primarily fitted over the left side of the user's shoe and the right portion is primarily fitted over the right portion of the user's shoe. As shown in FIGS. 2-5, the midstrap is arranged so that when the foot harness is deployed on a shoe or foot, the midstrap is circumferentially arranged around the mid-portion of the user's foot, just forward of the front of the ankle 58. In the example shown in FIGS. 2-5, the midstrap goes around the shoe generally just forward of the shoe heel 60 in the arch area, and over the top of the shoe just forward of the opening in the shoe through which the user puts the shoe on. The connecting plate 32 is arranged adjacent the area of the foot between the ball 62 of the foot and the heel 60 of the foot.

Referring again to FIGS. 6-8, the heel strap 28 includes a left heel portion 64 and a right heel portion 66. The right heel portion is connected generally at a right angle to the right section 56 of the midstrap and the left heel portion is connected generally at a right angle to the left section 54 of the midstrap. Both the left and right portions of the heel strap include coupling points. The heel strap coupling points, also referred to as the left and right side coupling points (68, 70), are provided just rearwardly of the midstrap. One of the heel strap portions also provides a heel coupling point 72. In the example shown in the figures, the left portion 64 of the heel strap 28 defines the heel coupling point 72 at its rear end. As with other coupling points, the left and right side coupling points (68, 70) and the heel coupling point 72 may be provided by a D-ring pivotally coupled with the heel strap by way of a section of strap material 42 deployed over the straight section 40 of the D-ring (which acts like an axle or pivot), and sewn to the surface of the heel strap to either side of the pivot portion of the D-ring. Alternative coupling member configurations may also be employed.

Depending on whether the exercise foot harness is deployed on a left or right foot of the user, the left and right coupling points (68, 70) may be oriented on the outside and inside of the foot or on the inside and outside of the foot, respectively. Examples of exercises that may be performed with the inside coupling point include a standing hip abduction with internal hip rotation and a standing hip abduction with external hip rotation. Examples of exercises that may be performed with the outside coupling point include a standing hip adduction with internal hip rotation and a standing hip adduction with external hip rotation. Examples of exercise that may be performed with the heel coupling point 72 include a standing knee extension and a standing hip flexion.

Referring to FIG. 7, as mentioned above, the foot harness is shown in its open non-deployed orientation. To attach the foot harness to a shoe, the foot harness may be first laid-out on floor as shown in FIG. 7. In this arrangement, the various coupling points 14 are located toward the floor when the straps are flat. The user places his or her foot on the harness as shown by the foot outline drawn in dashed-line. Given a typical size foot, in such a placement, the toe area 48 of the foot is placed over the toe strap 26 and the arch of the foot is placed over the midstrap 30. The connecting strap 32 provides, to some extent, a visual queue for the user to properly position his or her foot over the harness. For example, the user may center his or her foot over the connecting plate and place the ball 62 of his or her foot in proximity to the front of the connecting strap. This positions the foot so that the toe strap may be secured around the toe area of the shoe, the midstrap secured around the mid area of the shoe, and the heel strap coupled to the heel region 60 of the shoe.

After the shoe is placed on the foot harness, the user secures the harness 10 to the shoe 24. The user may first bring the left and right portions (35, 37) of the toe strap 26 up and around the left and right portions of the shoe, and then buckle the left and right portions together so that the strap surrounds the toe area 48 of the shoe. When buckled in this way, the top toe coupling point 34 will be arranged generally at the middle of the top of the toe area of the shoe. The bottom toe coupling 36 point will be arranged generally at the middle of the bottom of the toe area of the shoe.

Next, the user may secure the midstrap 30 of the harness to the shoe. If a flexible connecting strap 32 is employed, then the user may need to pull back on the harness once the toe strap is secured, to properly position the midstrap in relation to the foot. With some implementations, the connecting plate is rigid; thus, the midstrap will be properly located in relation to the foot after the toe strap is connected. Further, in some implementations an adjustable length connecting member may be employed so that even a wider variety of shoe sizes may be fitted with the exercise foot harness 10. In one particular implementation, the connecting strap or plate is about four inches in length and there is a separation of about 2¾″ between the toe and midstrap, which has been shown to allow the harness to fit a wide variety of shoe sizes.

To secure the midstrap to the user's shoe, the left and right portions (54, 56) of the midstrap are brought up and around the left and right portions, respectively, of the user's shoe, and connected together around the midportion of the shoe. In the implementation of FIGS. 2-8, the right portion of the midstrap, which has the loop portion 52 of the hook and loop fastener 46 arranged to face away from the shoe, is first placed along the top midportion of the shoe, then the left portion of the midstrap, which has the hook portion 50 of the hook and loop fastener arranged to face downward toward the shoe, is next placed over the right portion to properly engage the hook and loop fastener. The hook and loop fastener, as well as the buckles, allow a great range of adjustment for the harness so that each strap may be properly and firmly secured to the shoe.

After the midstrap is secured to the user's foot, the heel strap 28 is secured to the user's foot/shoe. The midstrap prearranges the left and right portions (64, 66) of the heel strap typically just above either side of the sole of the user' shoe, depending on the thickness of the sole. The left and right heel strap are then brought around the left and right sides of the heel, respectively, and buckled together at the rear of the heel. When buckled as such, the left and right coupling points (68, 70) are arranged generally below the front of the ankle and the heel coupling point 72 is arranged just above the rear center of the heel.

FIG. 9 is an isometric view of an alternative exercise foot harness, in accordance with one embodiment of the present invention. The foot harness shown in FIG. 9 is similar to the harness shown in FIGS. 2-8. Like the embodiment of FIGS. 2-6, the bottom toe coupling point 36 is provided with a strap sewn to the toe strap to define a connection channel as opposed to a D-ring. Unlike the embodiments of FIGS. 2-8, the top toe coupling point 34 is provided on the right portion 37 of the toe strap rather than the left portion.

FIGS. 10-12 illustrate a top view (how the harness is laid-out on a floor before securing to the shoe), a bottom view, and an isometric view, respectively, of an alternative embodiment of the foot harness 10, in accordance with the present invention. FIGS. 13-16 illustrate various views of the harness of FIGS. 10-12 deployed on a shoe. This embodiment is similar to the embodiment discussed above with regard to FIGS. 2-9. The embodiment of FIGS. 10-16 differs, however, in that it includes a coupling point centering buckle arrangement 78 operably associated with the toe strap 26 and the heel strap 28. The buckle arrangement makes it easier for the user to center the top toe coupling point at or near the midpoint of the width of the toe, and center the heel coupling point at or near the midpoint of the width of the back of the heel.

More particularly, the toe strap includes the left (first segment) 35 and the right (second segment) 37 strap portion. The right strap portion 37 has two strap segments (inside 74 and outside 76) and the buckling arrangement 78. The top toe coupling member 34 is connected with the outside strap. In FIGS. 10-12, the outside strap is shown connected with the inside strap (second segment) portion at a first ladder lock-type buckle 80. A second ladder lock-type buckle 82 is also secured at the outside end of the outside strap. When securing the harness to the shoe, the toe strap is wrapped around the toe area of the shoe, and the second buckle 82 is secured to the left portion 35 of the toe strap. To adjust the toe coupling point along any area of the toe region, the user may manipulate the first or second buckle and associated strap segments to adjust the effective length of the strap segments. Typically, the user will want to center the toe coupling point along the width of the front of the shoe.

Referring to FIG. 10, with regard to the heel strap28, a two ladder lock buckle arrangement 84 is also employed to provide for fitting of the heel strap and adjustment of the location of the heel coupling point 72. The left portion 64 or segment of the heel strap has two segments. The first segment 86 is connected with the midstrap and the second segment 88 is connected with the first segment with a ladder lock buckle 90. The second segment supports the heel coupling point 72. The right portion 66 has a second ladder lock buckle 92 at the end distal a connection with the midstrap. The second segment 88 has an end region adapted to adjustably engage the second ladder lock buckle 92.

To secure the heel strap around the heel, the second segment 88 of the left portion is attached to the right portion 66 using the second buckle 92. Further, by adjusting the first or second buckles (90, 92), the heel coupling point 72 located therebetween, may be positioned anywhere along the heel.

FIGS. 17-58 illustrate numerous alternative arrangements of an exercise foot harness conforming to various aspects of the present invention. Generally, in these alternatives some of the straps are rearranged or missing, some of the straps are replaced with shaped members or clips, and some of the straps include flexible resilient portions (i.e., “stretchable” or elastic segments). In addition, in some of the embodiments, the interconnections of the straps are performed with various different buckle-like pieces, hook and loop fasteners, clips, two-, three-, and four-way clips or buckles. Further, in some arrangements the exercise harness is configured from a single long strap or other material or the straps are separated from each other.

FIGS. 17 and 18 are a top view and a perspective view, respectively, of an alternative exercise foot harness 10, in accordance with aspects of the present invention, deployed on a typical athletic shoe 24. Particularly, the foot harness includes a toe strap 26, a midstrap 30, and a heel strap 28, similar in implementation as the straps shown with regard to FIGS. 2-16. The toe strap includes a top coupling point 34 and a bottom coupling point (not shown) arranged in about the same locations as the corresponding coupling points shown in FIGS. 2-16. In this implementation, the toe strap is coupled with a toe cup 94. The toe cup comprises a generally U-shaped partially flexible member with one leg 96 of the U connected with the top of the toe strap 26 and the other leg of the U (not shown) connected with the bottom of the toe strap. The connections to the toe strap are configured to align generally along the longitudinal midline of the shoe. At the apex of the U, the toe cup defines a split area 98 adapted to cup the tip of the shoe area. The split section defines two sections that straddle the tip of the shoe and thereby help to prevent side-to-side movement of the toe strap and to center the toe coupling points.

The toe strap 26 and midstrap 30 are connected by a first side strap 100 and a second side strap 102, with each side strap extending between the toe strap and the midstrap on either side of the shoe. To connect the toe strap to the first side strap and second side strap, the toe strap includes clips or buckles 104 defining an elongate aperture through which the side straps (100, 102) are routed. The clips are arranged along either side of the toe portion of the shoe when the harness is fitted on a shoe. The midstrap also includes two four-way clips 106 arranged along either side of the shoe when the harness is fitted on a shoe. The four-way clips define four elongate slots through which straps may be fed in various arrangements. When deployed on the shoe, the elongate apertures or slots are arranged so that there is a forward slot 108, rearward slot 110, upper slot 112, and lower slot 114. The clips on the midstrap are generally aligned with the clips on the toe strap. The side straps are routed between the forward slots 108 of the midstrap clips and the corresponding slots of the toe strap clips 104.

The side straps (100, 102) may include a hook and loop fastening arrangement 46 so that the length of the side straps and the separation between the toe strap and the midstrap may be adjusted. In one particular implementation, each side strap comprises a single length of strap with a hook section arranged along one end and a loop section arranged along the other end. The side strap is routed between the clip 104 on the toe strap and a corresponding forward slot on the four-way clip 106 on the midstrap so that the hook and loop portions are aligned for engagement. Depending on where along the length of the hook and loop portion, the hook and loop fastener is engaged, the length of the side straps may be adjusted to fit any length shoe and comfortably locate the midstrap along the shoe.

Alternatively, the side straps (100, 102) may be permanently attached between the clips (104, 106), and not include s hook and loop fastening arrangement. In such a configuration, the side straps define a single length of strap material. Each end of a strap is fed through the appropriate elongate aperture of the clip on the toe strap and the midstrap. The ends are then folded back toward the length of the strap between the clips, and sewn or otherwise fastened together. To provide adjustable length to the side straps, the side straps may include a section of flexible resilient material, such as an elastic material, along the length of the side strap. In this manner, the sides straps may be stretched to some degree when fitting the harness to the user's shoe, but will also provide constant tension on the length of the straps to provide a snug fit.

The midstrap 30 is similar to the midstrap discussed above with respect to FIGS. 2-16. The midstrap comprises a single length of strap with a hook section arranged along one end and a loop section arranged along the other end. The midstrap is routed through the upper and lower slots (112, 114) defined in the two four-clips 106. As such, the midstrap is arranged generally perpendicular to the side straps (100, 102). The midstrap is secured and appropriately tightened to the midportion of the shoe with the hook and loop fastening arrangement 46.

The heel strap comprises a right and left portion (64, 66) that extend around the back of the heel in a similar manner to the heel straps discussed with regard to FIGS. 2-16. The ends of the right and left portions of the heel strap are routed through the rearwardly oriented slots 110 of the four-way clips 106. The heel strap includes right and left side coupling points (68, 70) just rearward of the left and right four-way clips. The heel strap also includes a heel coupling point 72 arranged at the rear of the heel when the harness is worn by a user. The heel strap may include buckles, hook and loop fasteners, and/or elastic sections as described in various arrangements herein.

FIGS. 19 and 20 are a side view and a perspective view, respectively, of an alternative exercise foot harness 10, in accordance with aspects of the present invention, deployed on a typical athletic shoe 24. Particularly, the foot harness includes a toe strap 26, a midstrap 30, and a heel strap 28, similar in configuration as the straps shown with regard to other embodiments described herein. The toe strap is adapted to surround the toe area of the foot and position a top toe coupling point 34 and a bottom toe coupling point 36 as described elsewhere herein. In this embodiment, as with other embodiments, a connecting member extends between the toe strap and the midstrap. However, the connecting member 32 extends between the toe strap and the midstrap along the top portion of the shoe, typically where laces are found on a shoe. Alternatively, the connecting member may be arranged along the bottom of the shoe, such as is shown in FIGS. 2-16.

In the implementation of FIGS. 19 and 20, the midstrap comprises a separate lower strap 116 and an upper strap 118. The lower strap and upper strap are interconnected by two three-way clips 120 arranged on along either side of the shoe when the foot harness is worn by a user. The three-way clips have three slots arranged at about 120° to each other. The lower strap is adapted to be routed through the arch defined by the sole of many athletic shoes. Both three way clips are arranged so that one elongate aperture (slot) 122 is generally oriented toward the arch portion of the shoe. The lower strap is fed through these slots on each three-way clip. The lower strap may be of adjustable or non-adjustable length, may include a hook and loop fastening arrangement, may include a buckle, may include an elastic section, and/or may be sewn back on itself as described in various arrangements herein.

One of the slots 124 of each three-way clip is oriented generally upwardly and forwardly. The upper strap 118 is routed between these slots on each clip 120. The upper midstrap, in one particular implementation, includes a hook and loop fastening arrangement such as is described with respect to the midstrap of the harness of FIGS. 2-6, for example. Alternatively, buckles, elastic, and the like may be employed along the upper portion of the midstrap to provide a means by which the harness may be secured to the user's foot. The upper midstrap 118 extends between the two three-way clips over the top portion of the shoe.

The heel strap 28 extends around the heel area of the user's foot. Particularly, each three way clip defines rearwardly oriented elongate apertures or slots 126 through which the left and right portions of the heel strap are secured. Alternatively, the heel strap may be a single length strap piece routed between the left and right three-way clips. In the implementation of FIGS. 19 and 20, the heel strap includes a hook and loop type adjustable length feature to help fit the harness to a user's shoe. Alternatively, a single buckle or double buckle arrangement such is shown with regard to FIGS. 2-9 and 10-16, respectively, may be employed. Right 70, left 68, and heel 72 coupling points are arranged on the heel strap similarly to the harness described in FIGS. 2-16.

FIGS. 21 and 22 are a right side view and a perspective view, respectively, of an alternative exercise foot harness 10, in accordance with aspects of the present invention, deployed on a typical athletic shoe 24. The embodiment of FIGS. 21 and 22 is similar to the embodiment of FIGS. 19-20. Particularly, the embodiment of FIGS. 21 and 22 includes a midstrap 30 comprising an upper 118 and lower 116 strap interconnected with each other and with the heel strap via three-way clips 120 arranged generally above the arch area on either side of a typical athletic shoe. In addition, the toe strap 26 is connected with the midstrap by a connection member 32, either plate or strap, arranged along the lace area of an athletic shoe.

One difference between the embodiment of FIGS. 19 and 20 and that of FIGS. 21 and 22, is that the toe strap defines a single upper length and a split lower length. The split lower length includes a forward length 128 and a rearward length 130. For many athletic shoes, the toe area of the shoe includes an outwardly convex area on either side of the front of the shoe. The split lower length straddles this convex area on either side of the toe area of the shoe, and thereby provides a resistance to longitudinal (i.e., front and back) movement of the toe strap 26 when the user has a resistance mechanism coupled to either the lower 36 or upper 34 toe coupling points, or other coupling points. Also, rather than buckles or hook and loop fasteners, the upper strap 118 of the midstrap 30 and the heel strap 28 include elastic portions 132 to provide adjustable length to securely attach the harness to a user's shoe or foot.

FIGS. 23 and 24 are a right side view and a perspective view, respectively, of an alternative exercise foot harness 10, in accordance with aspects of the present invention. This embodiment includes a midstrap 30, two side straps 134, and a top connection strap 32. This embodiment also includes a toe cup 136 and a heel cup 138. The toe cup (or “toe member”) is a molded rubber, neoprene, or similar type material defining a cup conforming to the general shape of the toe portion of a typical athletic shoe. The side straps are connected with either side of the toe cup and are arranged to extend rearwardly of the toe cup. The top connection strap is connected with the top of the toe cup, and also arranged to extend rearwardly of the toe cup.

The heel cup (or “heel member”) is also a molded rubber, neoprene, or similar type material defining a cup conforming to the general shape of the heel portion of a typical athletic shoe. The bottom 140 of the heel cup is relatively flat. The perimeter open end of the heel cup defines a plane at an angle to the bottom of the cup. In the implementation of FIGS. 23-24, the plane defined by the open end of the cup is at about a 45 degree angle with respect to the flat bottom of the cup. The midstrap 30 is connected, on either side of the shoe, to the perimeter of the open end of the heel cup near the generally flat bottom. The midstrap is adapted to extend forwardly and over the top of the shoe near where the tongue of the shoe extends out of the shoe.

The side straps 134 are each connected between the toe cup 136 and the midstrap 30. The midstrap includes three-way clips 120 on the left and right sides of the shoe. The clip may slide along the midstrap. The left side strap is connected with the left clip and the right strap is connected with the right clip. The relationship between the side straps and the midstrap may be readily adjusted to provide custom fitting for the exercise foot harness. Additionally, each side strap may include an elastic section 132 to further provide for ease of securing the exercise foot harness to a user's shoe. In one implementation, each side strap defines a hook 142 at its rearward end that is adapted to engage a forwardly oriented slot defined in the three-way clips 120. To provide the sliding action, each clip defines opposing slots at about a right angle to the side strap slot. The midstrap is routed through each opposing slot so that the top surface of the strap is facing the bottom surface of the clip. Arranged as such, the clips are firmly held to the midstrap when force is applied to the left or right coupling points, but may be slid along the length of the midstrap when not in use through moderate force imparted by the user. Thus, the clips will tend to avoid sliding during typical use of the foot harness.

FIGS. 25 and 26 are a right side view and a perspective view, respectively, of another alternative embodiment of an exercise foot harness 10 in accordance with the present invention. This embodiment employs a toe cup 94 (or “toe member”) similar to that shown in FIGS. 17 and 18. The toe cup comprises a generally U-shaped partially flexible member generally configured so that the apex of the U fits around the toe portion of the shoe. At the apex of the U, the toe cup defines a split area 98 with two adjacent straps. The adjacent toe straps are arranged to fit on each side of the apex of the toe portion of the shoe to firmly hold the toe cup in position and to keep the toe cup from moving laterally (i.e. side-to-side) during use of the harness.

The toe cup further defines a Y-shaped rearwardly extending strap 144. The Y-shaped strap defines a single leg 146 and a pair of adjacent legs 148A, B. The single leg is connected with the toe cup. The pair of legs extend rearwardly from the single leg, and are each connected with a ring 150. More particularly, there are two front rings adapted to be located on either side of the shoe, just rearward of the toe area of the shoe, when the harness is worn. A midstrap 30 extends between each of the two front side rings. The midstrap is adapted to extend under the shoe when the harness is worn. The midstrap shown in FIGS. 25 and 26 does not include a top midstrap, which would extend over the shoe, and between the two front rings. Such a top midstrap, however, may be provided.

Side straps 134 (i.e. a left and right side strap) extend rearwardly from each front ring 150 and are connected with corresponding rear rings 152. About midway along the length of each side strap, an elongate aperture or slot 154 is provided along the bottom of the side straps. An arch strap 156 is fitted between the slots on the side straps. The arch strap includes an adjustable length feature. In the embodiment of FIGS. 25 and 26, the arch strap uses a hook and loop fastener adjustment mechanism for length adjustment. Several other length adjustment configurations as described herein may also be employed.

An adjustable length heel strap 28 extends between the two rear rings 152. When the harness is in use, the heel strap extends around the heel. The heel strap employs a hook and loop fastener type adjustment mechanism to allow the user to properly fit the harness to his or her shoe. A single or double buckle arrangement, or other length adjustment configurations may also be employed.

Several coupling points are provided in the foot harness shown in FIGS. 25 and 26. Particularly, the foot harness includes a top 34 and bottom 36 toe coupling point, left and right side coupling points (68, 70), and a heel coupling point 72. As with the other exercise foot harness embodiments described herein, numerous possible coupling point mechanisms are possible to provide the coupling points, such as D-rings, O-rings, carabiners, looped straps, hooks, clips, straps with buckles adapted to define a loop, etc.

FIGS. 27 and 28 illustrate a top view and a perspective view, respectively, of an alternative exercise foot harness, in accordance with aspects of the present invention, deployed on conventional athletic shoes. The exercise foot harness shown in FIGS. 27 and 28 includes a toe member 26, an arch strap 156, and a heel strap 28. The toe member, like other embodiments described herein, is adapted to encompass the toe portion of the shoe when the harness is worn. Unlike some of the other embodiments, the toe member of the harness of FIGS. 27 and 28 is a molded piece as opposed to a strap-like piece. As such, the toe member is more resilient, or less flexible, than some of the other embodiments described herein.

The front of the toe member defines a first circumferential opening 158. Note, the opening does not define a circle, per se, but is more of an elliptically shaped opening, which is the same for other embodiments discussed herein. The rear of the toe member defines a second circumferential opening 160. The toe member defines a cavity that widens from the front of the toe member rearwardly. As such, the toe member is adapted to fit over varying width shoes. For example, with a narrower shoe the toe member will fit more rearwardly of the front end of the shoe. In contrast, with a wider shoe, the toe member will fit more forwardly toward the front of the shoe.

Side straps 134 extend rearwardly, on either side of the shoe, between the toe member 26 and the heel strap 28. In the implementation shown, the toe member defines left and right rearwardly extending portions (162A, B). Each rearwardly extending portion defines a slot 164. The heel strap includes a two-slot buckle 166 at each of its ends. The first (top) slot of each buckle is aligned with corresponding side strap when the heel strap is fit to a shoe. As such, the side straps extend between the slots on the toe member and the top slot of the heel strap buckles. The second opening of each two-way buckle, is oriented generally downwardly. The arch strap 156 is fit through each of the lower slots and arranged under the shoe, generally through the archway, when the harness is worn.

The heel strap may include a hook and loop type fastener, single or double buckle arrangements, or other mechanisms to provide an adjustable length feature so that the harness may be properly fitted to each potential user. The side straps and the midstrap may include elastic portions, or may be completely elastic, to further provide flexibility when fitting the harness to various users. The embodiment shown in FIGS. 27 and 28 does not show a top portion of the midstrap; however, a top portion may be provided such as by providing a three-opening buckle at the ends of the heel straps with the third opening being oriented generally upward. The top portion of the midstrap may be arranged over the top of the shoe and between the third upwardly oriented openings of the three-opening buckles.

Several coupling points may be provided in the foot harness shown in FIGS. 27 and 28. Particularly, the foot harness includes a top and bottom (not shown) toe coupling point (34, 36), left and right side coupling points (68, 70), and a heel coupling point 72.

FIGS. 29 and 30 illustrate a top view and a perspective view, respectively, of yet another alternative exercise foot harness 10 in accordance with the present invention, deployed over a typical athletic shoe. The foot harness includes a molded neoprene toe member 26 similar to that shown in FIGS. 27 and 28. As such, the toe member defines a tapered cavity adapted to fit different width shoes or feet. Unlike the FIGS. 27 and 28 harness, the rearward extending side portions (162A, B) of the toe member extend rearwardly about the same distance as the side straps of the FIG. 27 embodiment. Thus, the side straps are not present in this embodiment. Instead, the rearwardly extending portions of the toe member are each directly connected with corresponding rings 150 oriented on each side of the shoe along the rear half of the length of the shoe. The top toe coupling point 34 is provided at about the top center of the toe member, and the bottom toe coupling point (not shown) is provided at about the bottom center of the toe member.

The heel strap 28 is also connected between each ring and adapted to go around the rear of the heel portion of the shoe. The heel strap may include a single or double buckle arrangement. The heel coupling point 72 is provided so as to be oriented at about the midpoint of the heel. The various buckle arrangements, or other adjustable length arrangements, not only allow the user to properly fit the exercise foot harness to different size shoes, but also allow the user to orient the heel coupling point.

An arch strap 156 is also connected between each ring. The arch strap shown in FIGS. 29 and 30 extends under the shoe, generally through the arch of the shoe, between the two side rings. A top strap, not shown, such as in a midstrap may further be provided to extend over the top of the shoe. In a midstrap arrangement or arch strap arrangement, a hook and loop fastener feature, e.g., Velcro™, facilitates proper fitting of the exercise foot harness to various size shoes and feet. The side coupling points (68, 70) are provided on the heel strap just rearwardly of the rings.

FIGS. 31 and 32 are a side right view and a perspective view, respectively, of an exercise foot harness, in accordance with one embodiment of the present invention, deployed over a typical athletic shoe. The harness of FIGS. 31 and 32 is similar to that shown in FIGS. 2-16. The FIGS. 31 and 32 embodiment includes a toe strap, a midstrap, and a heel strap. Unlike the FIGS. 2-16 embodiment, the toe straps (toe member) and heel strap (heel member) are molded neoprene or rubber members, i.e., not adjustable length. Each molded member is configured to properly locate the member around the heel and toe portion of the shoe. As such, the corresponding heel coupling point 72 and the toe coupling points (34, 36), may be properly located when the exercise foot harness is deployed on the user's foot.

Rather than employing a connecting plate, the harness of FIGS. 31 and 32 employs a pair of adjustable length side straps 134 extending between the sides of the toe strap and the forwardly extending end of the heel strap. The adjustable length of the side straps may be implemented with buckles, elastic, hook and loop arrangements, and the like. The side straps and the corresponding ends of the heel strap are coupled at a pair of rings 150 arranged along each side of the shoe when the harness is worn. The rings in this embodiment are shaped in the manner of one of the various Nautilus™ “shell” source identifiers. As with the embodiments of FIGS. 2-16, the midstrap has a portion that extends under the shoe between the rings, and a portion that extends over the shoe between the rings. The top portion is provided with a hook and loop fastener arrangement 46 so that the midstrap may be fitted to various width and style shoes. The midstrap may be a single length strap or comprise two or more sections of strap as discussed herein with regard to other midstraps and heel straps among others.

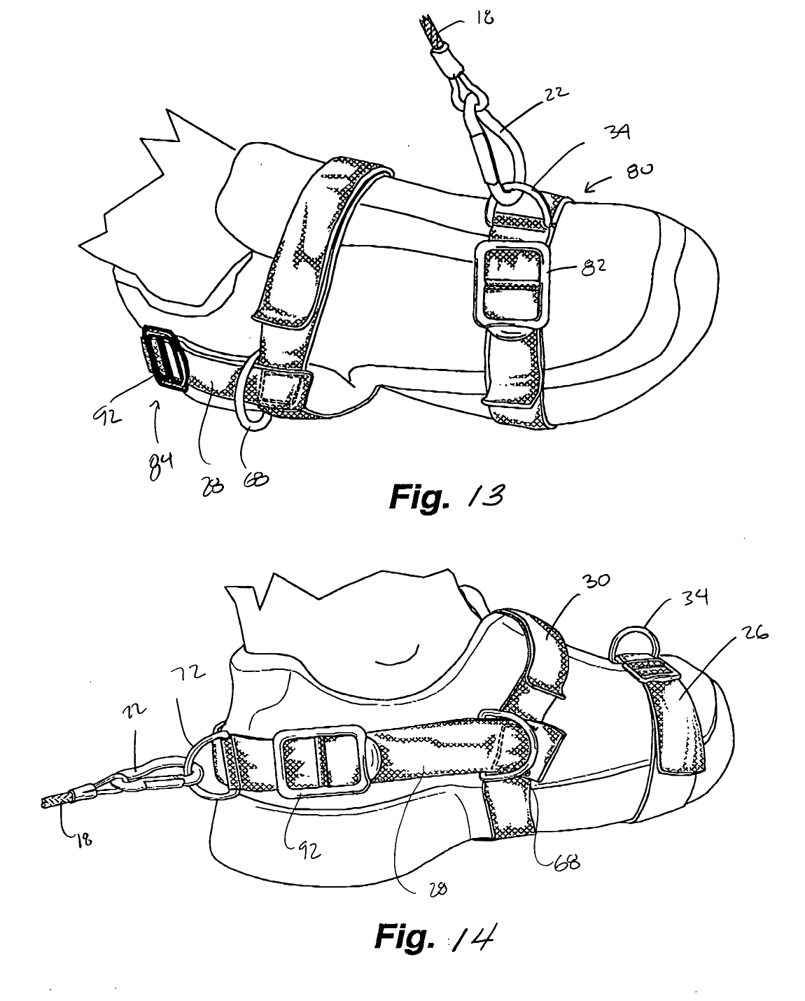

FIGS. 33 and 34 illustrate a right side view and a perspective view, respectively, of an alternative embodiment of an exercise foot harness 10, in accordance with aspects of the present invention, deployed over an athletic shoe. The embodiment of FIGS. 33-34, is a one-piece molded neoprene or other such material exercise foot harness. The one-piece exercise foot harness includes a toe member 26 and heel member 28. Both members define cavities adapted to fit around the toe and heel portion, respectively, of a typical athletic shoe. The toe member has an “open toe” which does not encompass the very front contour of the toe portion of the shoe. The toe member defines a varying circumference cavity with the narrower circumference oriented toward the front of the shoe and the wider circumference oriented toward the rear of the shoe. In this configuration, the toe member may be placed over the toe area of any size shoe. Depending on the width and shape of the toe portion of the shoe, the toe member will be secured to the region of the shoe where the dimensions of the toe member generally matches the dimensions of the toe portion of the shoe.

The heel member has an “open heel” which does not encompass the lower and rearmost contour of the heel portion of the shoe. The heel member defines a varying shape cavity adapted to fit over the heel area of a typical athletic shoe. Shaped as such, the heel member defines a generally U-shaped area. The concave apex region of the U is adapted to fit over the rear wall of the heel and each arm of the U is adapted to extend forwardly from each side of the heel. The heel member also includes a lower heel portion connected between the front of each leg of the U. The lower heel portion extends under the heel portion of a user's foot when the exercise harness is worn.

The heel portion and toe portion of the one-piece molded exercise foot harness are interconnected by opposing left and right side members 134. A midmember 30 extends around the midportion of the shoe. An upper segment 166 of the midmember extends over the top of the shoe between points about midway along the length of the side members. The midmember also includes a lower segment 168 adapted to extend under the shoe, respectively. The upper or lower member, or both, may be of an adjustable length, such as by employing a hook and loop fastener, one or more buckles, or other means. The lower member connects with a side member via a hook and loop fastener.

Coupling points (34, 36, 68, 70, 72) are provided at the upper portion of the toe member, the lower portion of the toe member, about midway along the length of each side member, and near the vertex of the apex region of the U-shaped heel member, respectively.

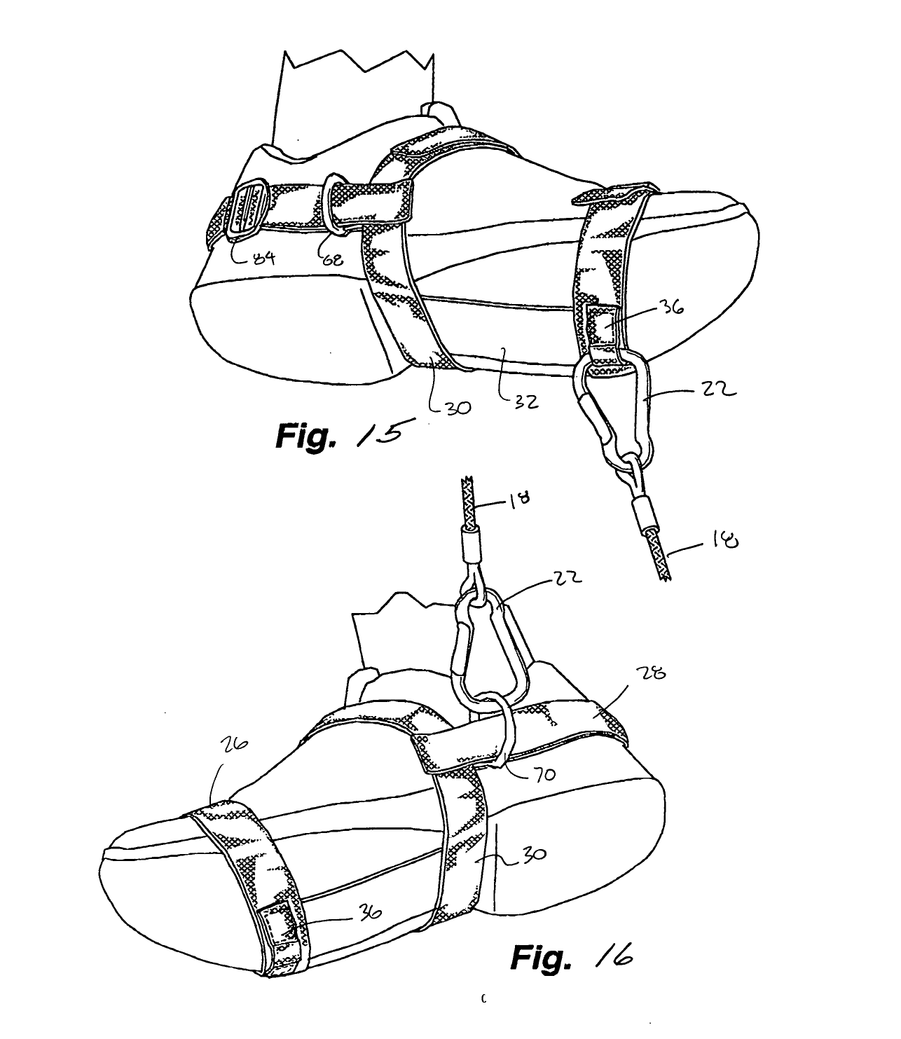

FIGS. 35 and 36 illustrate a right side view and a perspective view, respectively, of an alternative exercise foot harness 10, in accordance with one example of the present invention, deployed over an athletic shoe. This exercise foot harness includes an upper 116 and lower 118 midstrap, each interconnected with a molded heel member 28, but does not include a toe strap or member. The molded heel member is similar to the heel member discussed with respect to the exercise foot harness of FIGS. 33 and 34. As such, the heel member defines an “open heel” and is generally arranged in a U-shaped configuration. The apex region of the U, is adapted to conform to the contour of the heel region of a typical athletic shoe. The forward end of each forwardly extending leg of the U is connected with a ring 150. Oriented as such, each ring is located on either side of the shoe just forwardly of the midpoint of the shoe below where the laces on typical shoe may be found. The legs of the U may include an elastic portion 132.

The upper and lower portion of the midstrap are connected between the rings. The upper portion of the midstrap is adapted to fit over the top of a shoe or foot, and the lower portion is adapted to fit under the lower portion of the shoe. In this arrangement as well as some others discussed herein, the lower portion of the midstrap (sometimes referred to as an “arch strap”) may be particularly arranged to fit under the arch portion of the sole of a typical shoe. The arch area of the sole of many athletic shoes is concave; thus, orienting the lower portion of the midstrap within this concave area helps to keep the midstrap from moving forwardly or rearwardly during use of the exercise foot harness. In the area where the upper portion of the midstrap fits over the top of the shoe, the top of the shoe defines an angled surface. The interaction of the upper midstrap 116 with the upwardly oriented aspect of the top of the shoe helps to keep the upper portion of the midstrap from sliding backward along the shoe when rearward force is applied to the heel coupling point 72. The embodiment of FIGS. 35-36 includes a top coupling member 34 on the upper segment, a bottom coupling member 36 on the lower segment, and side coupling points (68, 70) on the legs of the U-shaped heel portion 28.

FIGS. 37 and 38 illustrate a right side view and perspective view, respectively, of an alternative exercise foot harness 10 in accordance with one example of the present invention, deployed over an athletic shoe. This exercise foot harness comprises a molded toe cup 136 and a molded heel cup 138. The toe cup has open end, similar to other “open toe” type toe cups discussed herein. The heel cup is not open-ended. Both the heel and toe cup are shaped to fit over the toe portion and heel portion of a typical athletic shoe.

A left side strap 134A extends between the left side of the toe cup and the left side of the heel cup. A right side strap 134B extends between the right side of the toe cup and the right side of the heel cup. The side straps may include elastic sections and may include adjustable length features such as buckles, hook and loop fasteners, pull clips, and the like. A midstrap 30 extends from the left front side of the heel cup to the right front side of the heel cup. The midstrap is of a length appropriate to fit over from one side of the heel cup around the top of the shoe Oust forward of the ankle) and back to the other side of the heel cup. The midstrap may also include adjustable length features such as buckles, a hook and loop fastener, pull clips and the like. An elastic arch strap 156 extends between one side of the midstrap under the arch of the shoe and to the opposing side of the midstrap. The elastic arch strap is also connected to each side strap 134A, B. The exercise harness of FIGS. 37 and 38 includes the various coupling points 14 of other embodiments discussed herein.

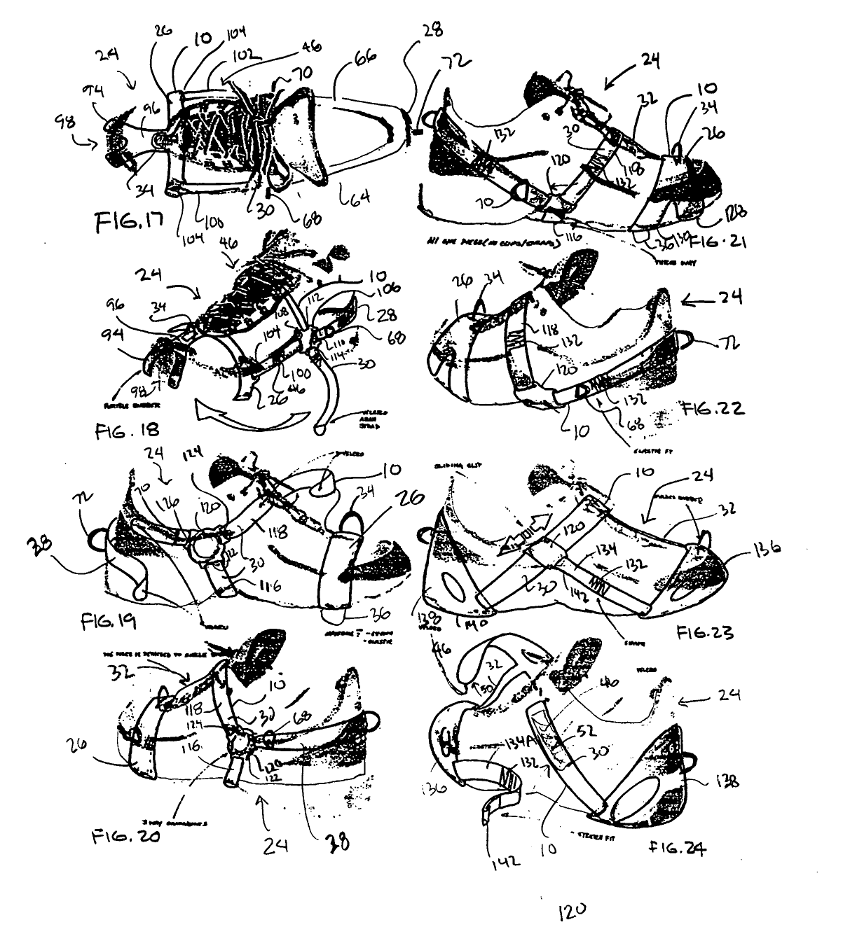

FIGS. 39 and 40 illustrate a right side view and a perspective view, respectively, of yet another alternative exercise foot harness 10, in accordance with aspects of the present invention, deployed over an athletic shoe. This embodiment includes a closed-end molded toe cup 136, a heel strap 28 and a separate, optional, midstrap 30. Upper and lower toe coupling points (34, 36) are provided on the toe cup. The heel strap is longer than other embodiments discussed herein, and is connected between each side of the toe cup. The heel strap includes one or more elastic segments 132 on either side of the shoe, and a buckle or pull clip 168 arranged near the rear of the heel portion of the shoe. A heel coupling point 72 is provided on the pull clip, or located on the strap, and oriented at or near the rear of the heel when the exercise foot harness is worn. Alternatively, a two buckle arrangement 78 may be employed in combination with the heel coupling point to provide an adjustment feature which easily allows the heel coupling point to be centered on the heel.

The midstrap in the harness example shown in FIGS. 39 and 40 is a separate piece. The midstrap employs a pull clip 168 so that it may be tightened onto any portion of the shoe, preferably in the area between the sole and where laces are found on many types of shoes. The midstrap includes right and left coupling points (68, 70). Note, because the midstrap is a separate piece, the right and left coupling points may be positioned on the left and right side of the shoe (such as is shown in most embodiments discussed herein) or may alternatively be positioned along other points of the shoe, such as at the top middle of the shoe and the bottom middle of the shoe, or just rearwardly of the upper and lower toe coupling points.

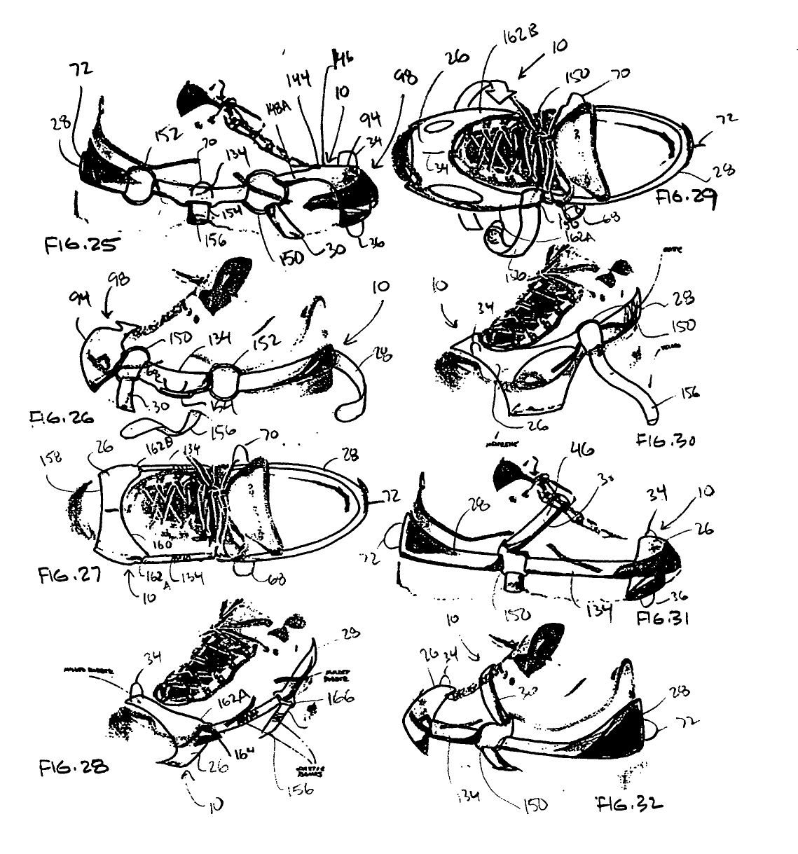

FIGS. 41 and 42 illustrate a top view and a perspective view, respectively, of the exercise foot harness shown in FIG. 43 attached to a typical athletic shoe. FIGS. 44 and 45 illustrate a top view and a perspective view of the exercise foot strap shown in FIG. 43 attached to a typical athletic shoe in a different arrangement than that shown in FIGS. 41 and 42. Referring first to FIG. 43, it can be seen that the exercise foot harness comprises two straps, a first 170 and a second 172 strap, that may be interconnected through a combination of hook and loop fasteners 46 and a four-way buckle/clip 106. Referring now to FIGS. 41 and 42, the first strap 170 may be configured to encompass the toe portion of a shoe, and the second strap 172 to encompass the mid portion of the user's shoe. When attached to a shoe, the four-way buckle is arranged rearwardly of the toe, on the top of the shoe generally aligned with the longitudinal center line of the shoe. The four elongate apertures (108, 110, 112, 114) of the four-way buckle are arranged at about a 45 degree angle with respect to the longitudinal center line.

The first strap 170 straddles the toe end of the shoe so that a segment of the strap is located along the sole of the shoe and transversely to the longitudinal center line of the shoe. The two ends of the first strap are fed into or aligned with the generally forwardly aligned slots (108, 114) of the four-way buckle. The second strap 172 is configured to have a segment located through the arch of the sole, generally transverse to the longitudinal center line of the shoe. The two ends of the second strap are fed into or aligned with the generally rearwardly aligned elongate apertures (110, 112) of the four-way buckle.

As can be seen in the diagram, the four-way buckle has opposing slots. For example, the rear and left oriented slot (112) is aligned with the right and front slot 108, and the rear and right oriented slot 110 is aligned with the front and left oriented slot 114. In such an arrangement, the ends of the first strap are aligned with the ends of the second strap via the buckle 106. Each end of the straps either include a hook or loop portion of a hook and loop fastener. As such, the ends of the first strap are adapted to secure via a hook and loop fastener to the aligned end of the second strap.

To secure the exercise foot harness of FIGS. 41-42 to a shoe or foot, the front strap cradles the toe portion of the shoe, and the second strap cradles the midportion of the shoe. The ends of the straps are fed through the four-way buckle and the hook and loop fasteners are engaged to secure the strap to the user's foot. Alternatively, as shown in FIGS. 44 and 45, the second strap 172 may be extended to cradle the heel portion of the shoe rather than the midportion of the shoe. A lower toe coupling point 36 is provided along the first strap, and a top toe coupling point 34 may be provided on the four-way buckle. Left and right side coupling points (68, 70) and a heel coupling point 72 may be provided at the appropriate points along the length of the second strap. When deployed around the midportion of the shoe, the heel coupling point may be arranged along the heel of the shoe to provide an alternative midsole coupling point.

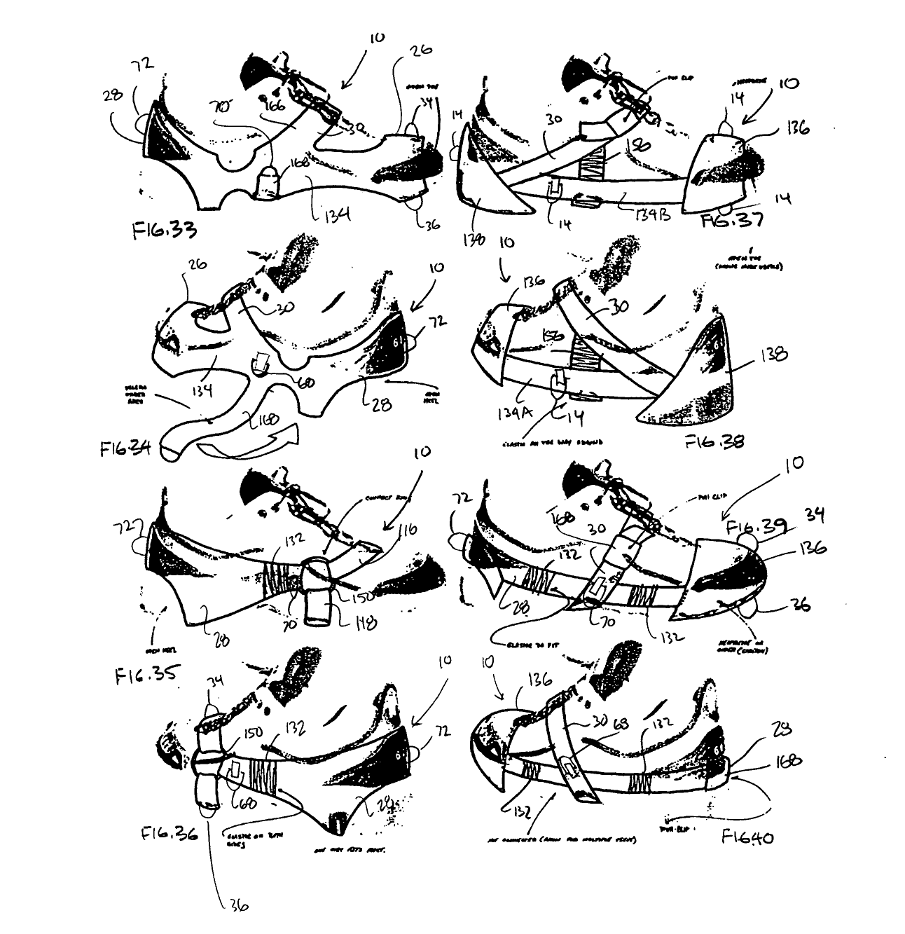

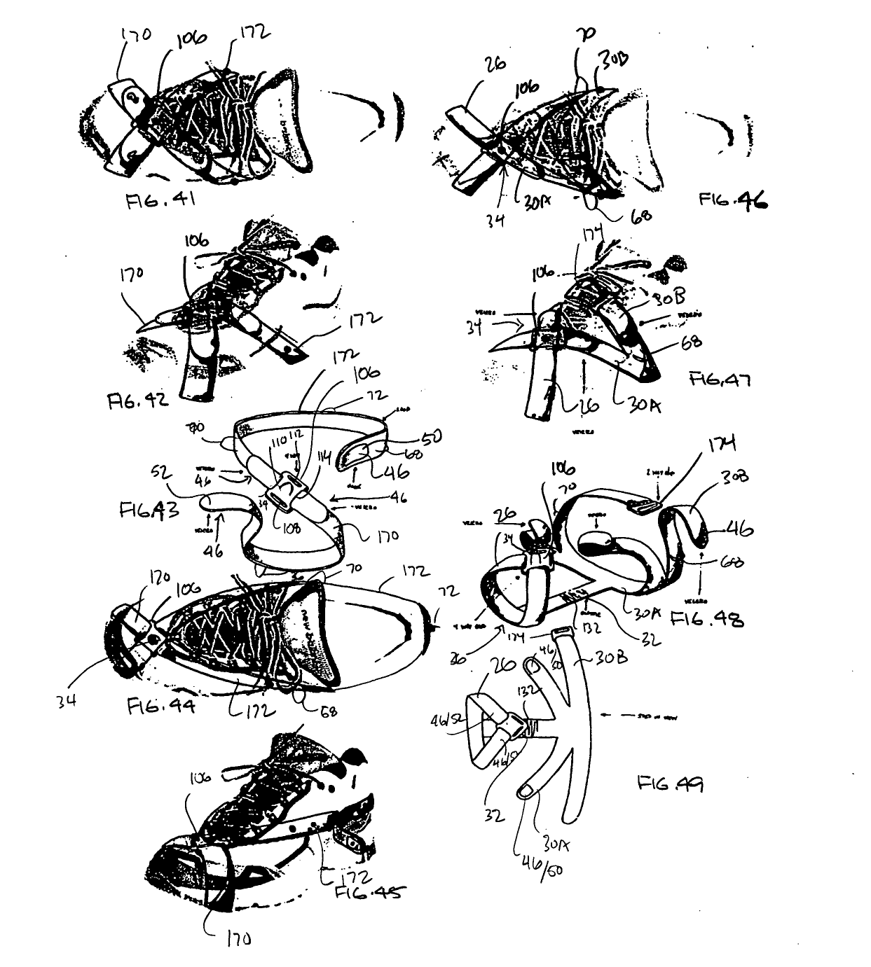

FIGS. 46 and 47 illustrate a top view and a perspective view, respectively, of an example of an exercise foot harness shown in FIGS. 48 and 49, deployed on a typical athletic shoe. Referring first to FIGS. 48 and 49, the exercise foot harness has a toe strap 26 connected with a first and a second midstrap (30A, 30B) via a connecting strap 32. The connecting strap may include an elastic section 132 to help fit the exercise harness to different sized shoes. The toe strap and first midstrap are adapted to interconnect with a four-way buckle 106 as discussed with reference to FIGS. 41-45. The second midstrap is adapted to extend over the top of the shoe rearwardly of the first midstrap. The second midstrap includes hook and loop fastener 46 in combination with a two-way clip/buckle to secure the second midstrap around the shoe. The embodiment of FIGS. 46-49 provides the top toe coupling point 34, bottom coupling point 36, side coupling points (68, 70), but does not provide a heel coupling point.

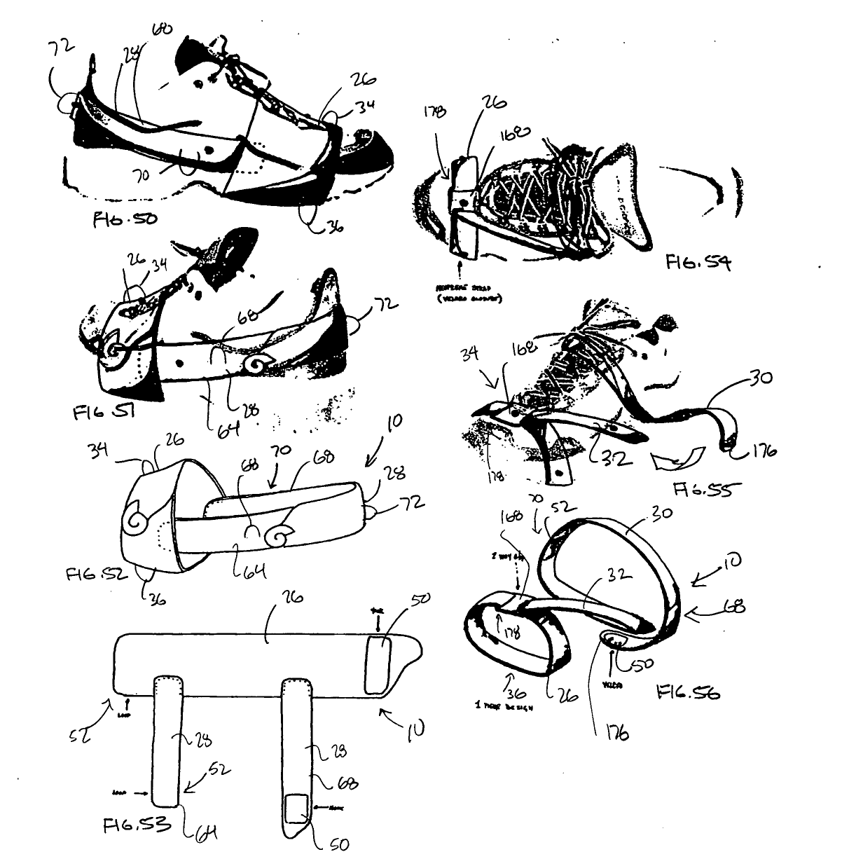

FIGS. 50 and 51 show a side view and a perspective view, respectively, of an alternative exercise foot strap 10 shown in FIGS. 52 and 53, deployed on a typical athletic shoe. Referring first to FIG. 53, a top view of the exercise foot harness before deployment is shown. The exercise foot strap comprises a toe strap 26 and a heel strap 28. The toe strap is single wide strap with a hook 50 (of a hook and loop fastener) along the top side at one end, and a loop 52 along the bottom side (or opposing side) at the other end. The heel strap includes two sections, a left section 64 and a right section 68. The left section includes a loop portion 52 of a hook loop fastener toward its rear end and on its bottom side. The right section includes a hook portion 50 of a hook and loop fastener toward its rear end and on its top side.

FIG. 52 shows the exercise foot strap with the various hook and loop fasteners engaged. FIGS. 50 and 51 show the exercise foot strap as in FIG. 52, and also deployed around a representative athletic shoe. When the hook and loop fasteners are engaged as such, the toe strap 26 defines a circumferential open-ended cavity adapted to fit around the toe portion of a shoe, and the heel strap 28 defines a U-shape adapted to fit around the back heel portion of the shoe. The heel strap is interconnected with the toe strap. Coupling points (34, 36, 68, 70, 72) may be located at the upper toe, lower toe, both sides, and heel area of the shoe, as discussed herein with reference to various embodiments.

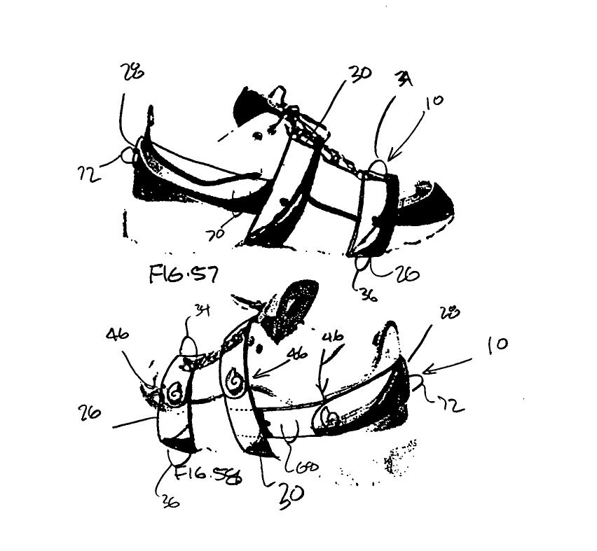

FIGS. 54-55 illustrate a top view and a perspective view, respectively, of an alternative exercise foot harness 10 shown in FIG. 56, deployed on a typical athletic shoe. The exercise foot harness of FIGS. 54-56, employs a single length of strap material to provide a toe strap 26, a midstrap 30, and a connecting strap 32. Referring to FIG. 56, at the rearward end 176 of the strap, a hook portion 50 of a hook and loop fastener is fixed to one surface of the strap. At a length roughly equivalent to the outside dimension of a typical shoe, the corresponding loop portion 52 of the hook and loop fastener is provided on the opposing surface of the strap. Arranged as such the rear portion of the strap may be fitted around the midportion of the shoe. The loop portion is long enough so that the length of the strap around the mid portion of the shoe may be adjusted to fit different size shoes.

Along the forward end of the strap 178, a two-way clip 168 is provided. The two-way clip is provided away from the end at a length roughly equivalent to the outside dimension of a typical shoe at the toe area. As such the front portion of the strap is fitted around the toe portion of the shoe, and the front end of the strap is fed into the two-way connector. The two-way clip may be moved along the length of the strap to adjust to different sized shoes. The portion of the strap between the two-way connector and the loop portion of the hook and loop fastener is fitted around the shoe and interconnects the toe strap and midstrap portions. The embodiment of FIGS. 54-56 may include the coupling points 34, 36, 68 and 70.

Finally, FIGS. 57 and 58 illustrate a right side view and a perspective view of an alternative exercise foot harness 10 in accordance with aspects of the present invention, deployed over a conventional athletic shoe. This embodiment is similar to that discussed above with respect to FIGS. 2-6. However, the embodiment of the FIGS. 57 and 58 employs hook and loop fasteners 46 on the toe strap and the heel strap in place of the buckle arrangements shown in FIGS. 2-6. In addition, the ends of the toe strap, midstrap, and heel strap are provided with decorative or source identifying feature, such as the well recognized Nautilus™ Shell™ source identifier.

Although preferred embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the embodiments of the present invention, and do not create limitations, particularly as to the position, orientation, or use of the invention unless specifically set forth in the claims. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

In some instances, components are described with reference to “ends” having a particular characteristic and/or being connected to another part. However, those skilled in the art will recognize that the present invention is not limited to components which terminate immediately beyond their points of connection with other parts. Thus, the term “end” should be interpreted broadly, in a manner that includes areas adjacent, rearward, forward of, or otherwise near the terminus of a particular element, link, component, member or the like. In methodologies directly or indirectly set forth herein, various steps and operations are described in one possible order of operation, but those skilled in the art will recognize that steps and operations may be rearranged, replaced, or eliminated without necessarily departing from the spirit and scope of the present invention. It is intended that all matter contained in the above description or shown in the accompanying drawings shall be interpreted as illustrative only and not limiting. Changes in detail or structure may be made without departing from the spirit of the invention as defined in the appended claims.

It will be recognized by those skilled in the art, that various elements discussed with reference to the various embodiments may be interchanged to create entirely new embodiments coming within the scope of the present invention. For example, the toe member discussed with reference to FIGS. 27 and 28, may replace the toe strap of the FIG. 2 embodiment. Buckles may be replaced with hook and loop fasteners, where a single buckle arrangement is shown, a double ladder lock buckle arrangement may be substituted, etc. It is intended that all matter contained in the above description or shown in the accompanying drawings shall be interpreted as illustrative only and not limiting. Changes in detail or structure may be made without departing from the spirit of the invention as defined in the appended claims.

1. An exercise foot harness comprising:
a toe strap defining a first segment and a second segment, the toe strap further comprising at least one coupling member adapted to interconnect the first segment and the second segment, the toe strap further comprising at least one first coupling point;
a connecting member coupled with the toe member;
a midstrap coupled with the connecting member, the midstrap defining a third segment and a fourth segment, the midstrap further comprising at least one second coupling member adapted to interconnect the third segment and the fourth segment; and
a heel strap coupled with the midstrap, the heel strap defining a fifth segment and a sixth segment, the heel strap further comprising at least one third coupling member adapted to interconnect the fifth segment and the sixth segment, the heel strap further comprising at least one second coupling point.

2. The exercise foot harness of claim 1 wherein:
the toe strap is configured to be secured around a toe portion of a typical athletic shoe;
the midstrap is configured to be secured around a midportion of a typical athletic shoe; and
the heel strap is configured to be secured around a heel portion of a typical athletic shoe.

3. The exercise foot harness of claim 1 wherein the at least one coupling member is selected from the group consisting of a buckle, a ladder lock buckle, a hook and loop fastener, a clip, and a snap lock.

4. The exercise foot harness of claim 1 wherein the at least one coupling member comprises means for coupling the first segment with the second segment.

5. The exercise foot harness of claim 1 wherein the toe strap further comprises a first adjustment segment supporting the at least one first coupling point.

6. The exercise foot harness of claim 5 wherein the first adjustment segment is arranged adjacent the second segment.

7. The exercise foot harness of claim 6 wherein the at least one coupling member comprises a first ladder lock buckle adapted to interconnect the second segment and the first adjustment segment and further comprising a second ladder lock buckle adapted to interconnect the first adjustment segment and the first segment.

8. The exercise foot harness of claim 7 wherein the first ladder lock buckle and the second ladder lock buckle are arranged to provide adjustment for the at least one first coupling point.

9. The exercise foot harness of claim 1 wherein the at least one second coupling member is selected from the group consisting of a buckle, a ladder lock buckle, a hook and loop fastener, a clip, and a snap lock.

10. The exercise foot harness of claim 1 wherein the at least one second coupling member comprises means for coupling the first segment with the second segment.

11. The exercise foot harness of claim 1 wherein the at least one third coupling member is selected from the group consisting of a buckle, a ladder lock buckle, a hook and loop fastener, a clip, and a snap lock.

12. The exercise foot harness of claim 1 wherein the at least one third coupling member comprises means for coupling the first segment with the second segment.

13. The exercise foot harness of claim 1 wherein the heel strap further comprises a seventh segment supporting the at least one second coupling point.

14. The exercise foot harness of claim 13 wherein the seventh segment is arranged adjacent the sixth segment.

15. The exercise foot harness of claim 14 wherein the at least one third coupling member comprises a third ladder lock buckle adapted to interconnect the sixth segment and the seventh segment and further comprising a fourth ladder lock buckle adapted to interconnect the seventh segment and the fifth segment.

16. The exercise foot harness of claim 15 wherein the third ladder lock buckle and the fourth ladder lock buckle are arranged to provide adjustment for the at least one second coupling point.

17. The exercise foot harness of claim 1 wherein:
the at least one first coupling point comprises a top toe coupling point and a bottom toe coupling point;
the at least second coupling point comprises a left side coupling point, a right side coupling point, and a heel coupling point.

18. The exercise foot harness of claim 17 wherein:
the top toe coupling point is arranged generally along the top of the toe region of a typical athletic shoe when the exercise foot harness is deployed;
the bottom toe coupling point is arranged generally along the bottom of the toe region of a typical athletic shoe when the exercise foot harness is deployed;
the left side coupling point is arranged generally along the left side of a typical athletic shoe when the exercise foot harness is deployed;
the right side coupling point is arranged generally along the right side of a typical athletic shoe when the exercise foot harness is deployed; and
the heel coupling point is arranged generally along the heel of a typical athletic shoe when the exercise foot harness is deployed.

19. The exercise foot harness of claim 1 wherein the at least one coupling point is selected from the group consisting of a ring, a D-ring, an O-ring, a strap, a hook, a loop, a carabiner, and an eyelet.

20. The exercise foot harness of claim 1 wherein the at least one second coupling point is selected from the group consisting of a ring, a D-ring, an O-ring, a strap, a hook, a loop, a carabiner, and an eyelet.

21. An exercise foot harness adapted for being secured to an athletic shoe, the athletic shoe including a toe region, a heel region, and a mid region between the toe region and the heel region, comprising:
means for providing at least one coupling point at the toe region of an athletic shoe;
means for providing at least one second coupling point at the heel region of an athletic shoe; and
means for securing the exercise foot harness to the mid region of an athletic shoe.

22. The exercise foot harness of claim 22 further comprising means for interconnecting the means for providing at least one coupling point to the toe region of an athletic shoe with the means for securing the exercise foot harness to the mid region of an athletic shoe.

23. The exercise foot harness of claim 22 wherein the means for providing at least one second coupling point to the heel region of an athletic shoe is coupled with the means for securing the exercise foot harness to the mid region of an athletic shoe.

24. An exercise foot harness comprising:
a toe member adapted to engage a toe portion of a typical athletic shoe;
a heel member adapted to engage a heel portion of a typical athletic shoe;
at least one interconnecting member coupled between the toe member and the heel member; and
a plurality of coupling points.

25. The exercise foot harness of claim 24 further comprising:
a midmember adapted to engage a midportion of a typical athletic shoe.

26. The exercise foot harness of claim 25 wherein the midmember comprises a first strap adapted to engage the arch region of a typical athletic shoe.

27. The exercise foot harness of claim 25 wherein the midmember comprises a second strap adapted to engage the lace region of a typical athletic shoe.

28. The exercise foot harness of claim 25 wherein the at least one interconnecting member comprises a first side strap coupled between the toe member and the heel member and a second side strap coupled between the heel member and the toe member.

29. The exercise foot harness of claim 24 wherein the toe member comprises at least one strap including a length adjustment feature.

30. The exercise foot harness of claim 29 wherein the heel member comprises at least one second strap including a second length adjustment feature.

31. The exercise foot harness of claim 30 wherein the at least one interconnecting member comprises at least one third strap including a third length adjustmemt feature.

32. The exercise foot harness of claim 24 wherein the at least one toe member comprises a molded cavity adapted to engage the toe portion of the typical athletic shoe.

33. The exercise foot harness of claim 32 wherein the at least one heel member comprises a second molded cavity adapted to engage the heel portion of a typical athletic shoe.

34. An exercise foot harness comprising:
a first member adapted to engage a front portion of a typical athletic shoe;
a second member adapted to engage a rear portion of a typical athletic shoe; and
at least one coupling point.

35. The exercise foot harness of claim 34 wherein the first member comprises a toe member with a first coupling point and a second coupling point.

36. The exercise foot harness of claim 34 wherein the second member comprises a heel member with a third coupling point and a fourth coupling point.

37. The exercise foot harness of claim 36 wherein the first member comprises a midmember.

38. The exercise foot harness of claim 34 the first member and the second member are provided by a single strap.

39. The exercise foot harness of claim 34 further comprising at least one interconnecting member between the first member and the second member.

40. The exercise foot harness of claim 39 further comprising a third member adapted to engage a rear portion of a typical athletic shoe.

41. The exercise foot harness of claim 34 wherein the front portion of a typical athletic shoe comprises a toe area.

42. The exercise foot harness of claim 34 wherein the rear portion of a typical athletic shoe comprises the lacing area.

43. The exercise foot harness of claim 34 wherein the rear portion of a typical athletic shoe comprises the heel area.

44. The exercise foot harness of claim 34 wherein the rear portion of a typical athletic shoe comprises a region between and including the lacing area and the heel area.
